SEQUENCE LISTING (1 sequence)

SEQ ID NO: 1 - protein, 413 aa, Triticum aestivum
MGKHSATLCGLLVVVLCLASSLAQAQILFQGFNWESWKTQGGWYKFMQGKVEEIASTGATHVWLPPPSQSVSPEGYLPGQLYNLNSKYGSGADLKSLIQAFRGKNISCVADIVINHRCADKKDGRGVYCIFEGGTSDNRLDWGPDEICSDDTKYSNGRGHRDTGGGFDAAPDIDHLNPRVQRELSAWLNWLKTDLGFDGWRLDFAKGYSAAMAKIYVDNSKPAFVVGELYDRDRQLLANWVRGVGGPATAFDFPTKGVLQEAVQGDLGRMRGSDGKAPGMIGWMPEKTVTFIDNHDTGSTQRLWPFPSDKVMQGYAYILTHPGIPCIFYDHVFDWKLKQEITALATVRSRNGIHPGSTLDILKAEGDLYVAKIGGKVITKIGSRYNIGDNVIPSGFKIAAKGNNYCVWEKSGL

(19) United States
(12) Patent Application Publication
(10) Pub. No.: US 2026/0251656 A1
(43) Pub. Date: Aug. 27, 2026

(54) SUBSTRATE AND LATERAL FLOW DEVICE FOR DETECTING ALPHA-AMYLASE ACTIVITY

(71) Applicant: ENVIROLOGIX, INC., Portland, ME (US)

(72) Inventors: Adam Johnson, Portland, ME (US); Lawrence Pessolano, Portland, ME (US)

(73) Assignee: ENVIROLOGIX, INC., Portland, ME (US)

(21) Appl. No.: 19/570,827

(22) Filed: Mar. 18, 2026

Related U.S. Application Data
(63) Continuation of application No. PCT/US2024/048649, filed on Sep. 26, 2024.
(60) Provisional application No. 63/585,701, filed on Sep. 27, 2023.

(51) Int. Cl. G01N 33/573; G01N 33/533; G01N 33/543; G01N 33/58

(52) U.S. Cl. CPC G01N 33/573; G01N 33/533; G01N 33/54388; G01N 33/582; G01N 2333/932

(57) ABSTRACT

Lateral flow devices (LFDs) and insoluble fluorescence (FAM)-labeled starch substrates, compositions comprising the same, and methods for use thereof in the measurement of alpha amylase activity in a sample.

### CROSS-REFERENCE TO RELATED APPLICATION

This application is a continuation under 35 U.S.C. § 111 (a) of PCT International Patent Application No. PCT/US2024/048649, filed Sep. 26, 2024, designating the United States and published in English, which claims priority to and the benefit of U.S. Provisional Application No. U.S. 63/585,701, filed Sep. 27, 2023, the entire contents of each of which are incorporated by reference herein.

### SEQUENCE LISTING

The instant application contains a Sequence Listing which has been submitted electronically in XML format and is hereby incorporated by reference in its entirety. The Sequence Listing XML file, created on Apr. 22, 2026, is named 167665-012002_US_SL.xml and is 2,336 bytes in size.

### BACKGROUND OF THE INVENTION

Alpha-amylase is considered to be one of the primary enzymes responsible for starch degradation in cereals and particularly wheat. During grain germination, α-amylase initiates the conversion of starch into simple sugars to fuel embryo and coleoptile growth in the first few days of germination. Also, late maturity alpha-amylase and pre-harvest sprouting are both recognized as environmentally induced grain quality defects resulting from abnormally high levels of α-amylase. High levels of alpha amylase in a grain are an indicator of poor-quality grain and, consequently, of poor quality products produced from the grain.

The Falling Numbers method is widely employed to detect alpha-amylase activity in cereals. However, it has several limitations, including sampling variability, high cost, labor intensiveness, and the destructive nature of the test. Faster, cheaper, and more accurate alternatives could improve breeding for resistance to preharvest sprouting and late maturity alpha-amylase and could preserve the value of wheat grain by avoiding inadvertent mixing of high- and low-Falling Numbers grain by enabling testing at more stages of the value stream, including at harvest, delivery, transport, storage, and milling.

Therefore, there is a present need for improved methods for measuring alpha-amylase activity in a sample.

### SUMMARY OF THE INVENTION

The disclosure features lateral flow devices (LFDs) and insoluble fluorescence (FAM)-labeled starch substrates, compositions comprising the same, and methods for use thereof in the measurement of alpha amylase activity in a sample.

In one aspect, the disclosure features a method for producing a substrate for use in detecting alpha amylase activity in a liquid sample. The method involves a) covalently linking a fluorescein molecule (FAM) to a natural starch molecule to yield a FAM-labeled starch substrate. The method further involves b) suspending the FAM-labeled starch substrate in one or more aqueous solutions one or more times. Each suspending is followed by separation of the FAM-labeled starch substrate from the aqueous solution. The method also involves c) suspending the FAM-labeled starch substrate in a volatile organic solvent followed by drying of the FAM-labeled starch substrate to remove the volatile organic solvent.

In another aspect, the disclosure features a method for producing a substrate for use in detecting alpha amylase activity in a liquid sample. The method involves a) covalently linking a fluorescein molecule (FAM) to a corn starch molecule using adipic acid dihydrazide to yield a FAM-labeled starch substrate. The method further involves b) suspending the FAM-labeled starch substrate in purified water between 5 and 10 times. Each suspending is followed by separation of the FAM-labeled starch substrate from the purified water using centrifugation. One or more of the washes of the FAM-labeled starch substrate in purified water involves heating the purified water containing the FAM-labeled starch substrate suspended therein to a temperature of between about 40° C. and 80° C. for between about 5 min and 30 min prior to separating the FAM-labeled starch substrate from the purified water. The method further involves c) suspending the FAM-labeled starch substrate in an aqueous solution with a pH of between 7 and 8 containing a Tris buffer and between 2 and 50 nM Ca²⁺ ions. The method also involves incubating the suspension at a temperature of between about 40° C. and 80° C. for between about 5 min and 30 min prior to separating the FAM-labeled starch substrate from the aqueous solution using centrifugation. The method further involves d) suspending the FAM-labeled starch substrate in methanol followed by drying of the FAM-labeled starch substrate to remove the methanol.

In another aspect, the disclosure features a method for measuring alpha-amylase activity in a grain sample. The method involves a) preparing an aqueous mixture containing the grain sample and the FAM-labeled starch substrate prepared according to the method of any aspect of the disclosure, or embodiments thereof, under conditions suitable for any alpha-amylase protein present in the grain sample to degrade the FAM-labeled starch substrate and thereby produce water-soluble FAM-labeled starch fragments in the aqueous mixture. The method also involves, b) separating undegraded FAM-labeled starch substrate and the grain sample from the aqueous mixture to yield a soluble fraction. The method further involves c) quantifying the amount of FAM-labeled starch fragments in the soluble fraction.

In another aspect, the disclosure features a method for measuring alpha-amylase activity in a grain sample. The method involves a) contacting the grain sample with an aqueous solution at a mass to volume ratio of grain sample to aqueous solution of about 5:1 for between 15 seconds and 1 minute to extract alpha-amylase polypeptides from the grain sample. The method also involves b) separating the aqueous solution from the grain sample to yield an extract solution. The method also involves c) adding between 3 mg/mL and 10 mg/mL of the FAM-labeled starch substrate of any aspect of the disclosure, or embodiments thereof, to the extract solution and incubating the resulting mixture at a temperature of between about 65° C. and 75° C. for between 1 minute and 2 minutes under conditions suitable for an alpha-amylase protein present in the extract solution to degrade the FAM-labeled starch substrate prior to stopping the reaction to thereby produce water-soluble FAM-labeled starch fragments in the extract solution. The method also involves d) quantifying the amount of FAM-labeled starch fragments in the extract solution using a lateral flow device (LFD) containing an anti-fluorescein antibody conjugated to a colloidal gold particle, a test line, and a control line. In the lateral flow device, i) the test line contains a polypeptide conjugated to fluorescein; and ii) the control line contains an antibody capable of binding to the anti-fluorescein antibody. LFD further contains i) a first portion containing a site for application of a liquid sample, a liquid-permeable medium, and the anti-fluorescein antibody conjugated to the colloidal gold particle; ii) a second portion in capillary communication with the first portion and containing a liquid-permeable medium containing, in order of distance from the first portion, the test line and the control line; and iii) a third portion in capillary communication with the second portion and containing a liquid-permeable medium.

In another aspect, the disclosure features a FAM-labeled starch substrate produced by the method of any aspect of the disclosure, or embodiments thereof.

In another aspect, the disclosure features a method for measuring alpha-amylase activity in a grain sample. The method involves a) contacting the grain sample with an aqueous solution to extract alpha-amylase polypeptides from the grain sample. The method further involves b) separating the aqueous solution from the grain sample to yield an extract solution. The method also involves c) adding the FAM-labeled starch substrate of any aspect of the disclosure, or embodiments thereof, to the extract solution under conditions suitable for an alpha-amylase protein present in the extract solution to degrade the FAM-labeled starch substrate and thereby produce water-soluble FAM-labeled starch fragments in the extract solution. The method also involves d) quantifying the amount of FAM-labeled starch fragments in the extract solution.

In another aspect, the disclosure features a kit suitable for use in the method of any aspect of the disclosure, or embodiments thereof, where the kit contains the FAM-labeled starch substrate of any aspect of the disclosure, or embodiments thereof, disposed within a container.

In another aspect, the disclosure features a composition containing a volatile organic substrate and a natural starch molecule covalently linked to a fluorescein molecule by an adipic acid dihydrazine linker.

In another aspect, the disclosure features a tablet containing microcrystalline cellulose and an insoluble fluorescein-labeled starch substrate containing a natural starch molecule covalently linked to a fluorescein molecule by an adipic acid dihydrazine linker.

In another aspect, the disclosure features a lateral flow device (LFD) containing an anti-fluorescein antibody conjugated to a colloidal gold particle, a test line and a control line. The test line contains a polypeptide conjugated to fluorescein. The control line contains an antibody capable of binding to the anti-fluorescein antibody.

In any aspect of the disclosure, or embodiments thereof, covalently linking the FAM to the natural starch molecule involves a) contacting the natural starch molecule in aqueous solution with carbonyldiimidazole to activate accessible hydroxyl groups. The covalently linking further involves b) contacting the FAM with adipic acid dihydrazine (AAD) in an aqueous solution containing 1-Ethyl-3-(3-dimethylaminopropyl) carbodiimide (EDC) so that a carboxyl group of the fluorescein becomes covalently linked to the AAD. The covalently linking also involves c) combining the products of a) and b) to allow the AAD bound to the FAM to react with the activated accessible hydroxyl groups of the natural starch molecule to yield the FAM-labeled starch substrate.

In any aspect of the disclosure, or embodiments thereof, the natural starch is corn starch or wheat starch.

In any aspect of the disclosure, or embodiments thereof, the one or more aqueous solutions contains purified water.

In any aspect of the disclosure, or embodiments thereof, b) involves washing the FAM-labeled starch substrate in purified water between 5 and 10 times. Each washing involves suspending the FAM-labeled starch substrate in the purified water and subsequently separating the FAM-labeled starch substrate from the purified water using centrifugation or filtration.

In any aspect of the disclosure, or embodiments thereof, at least one of the aqueous solution contains between 2 and 50 mM Ca²⁺ ions.

In any aspect of the disclosure, or embodiments thereof, one or more of the washes of the FAM-labeled starch substrate in purified water involves heating the purified water containing the FAM-labeled starch substrate suspended therein to a temperature of between about 40° C. and 80° C. for between about 5 min and 30 min prior to separating the FAM-labeled starch substrate from the purified water.

In any aspect of the disclosure, or embodiments thereof, b) further involves washing the FAM-labeled starch substrate one or more times in the aqueous solution containing Ca²⁺ ions after the between 5 and 10 washes of the FAM-labeled starch substrate in purified water by suspending the FAM-labeled starch substrate in the aqueous solution containing Ca²⁺ ions and subsequently separating the FAM-labeled starch substrate from the purified water using centrifugation or filtration. In any aspect of the disclosure, or embodiments thereof, the aqueous solution containing Ca²⁺ ions contains a buffer.

In any aspect of the disclosure, or embodiments thereof, the buffer contains a primary amine. In any aspect of the disclosure, or embodiments thereof, the buffer is a Tris buffer.

In any aspect of the disclosure, or embodiments thereof, one or more of the washes of the FAM-labeled starch substrate in the aqueous solution containing the Ca²⁺ ions involves heating the aqueous solution containing the Ca²⁺ ions and containing the FAM-labeled starch substrate suspended therein to a temperature of between about 40° C. and 80° C. for between about 5 min and 30 min prior to separating the FAM-labeled starch substrate from the aqueous solution containing the Ca²⁺ ions.

In any aspect of the disclosure, or embodiments thereof, c) further involves heating the volatile organic solvent containing the FAM-labeled starch substrate suspended therein to a temperature of between 40° C. and 80° C. for a period of between about 5 min and 30 min prior to removal of the volatile organic solvent.

In any aspect of the disclosure, or embodiments thereof, suspending the FAM-labeled starch substrate in a) and/or b) involves sonication.

In any aspect of the disclosure, or embodiments thereof, c) takes place after b).

In any aspect of the disclosure, or embodiments thereof, the method further involves sieving the dried FAM-labeled starch substrate.

In any aspect of the disclosure, or embodiments thereof, the average particle size of the FAM-labeled starch substrate is between 5 μm and 200 μm.

In any aspect of the disclosure, or embodiments thereof, the method further involves preparing tablets containing the FAM-labeled starch substrate. In any aspect of the disclosure, or embodiments thereof, the tablets contain between 40 mg and 160 mg of the FAM-labeled starch substrate. In any aspect of the disclosure, or embodiments thereof, the tablets contain microcrystalline cellulose.

In any aspect of the disclosure, or embodiments thereof, the concentration of the grain sample in the aqueous mixture is from about 0.1 g/mL to about 1 g/mL. In any aspect of the disclosure, or embodiments thereof, the volume to mass ratio of the grain sample to the aqueous solution is from about 2:1 to 10:1. In any aspect of the disclosure, or embodiments thereof, the volume to mass ratio of the grain sample to the aqueous solution is about 5:1. In any aspect of the disclosure, or embodiments thereof, the concentration of the FAM-labeled starch substrate added to the aqueous mixture is from about 0.2 mg/mL to about 500 mg/mL.

In any aspect of the disclosure, or embodiments thereof, the concentration of the FAM-labeled starch substrate added to the extract solution is from about 3 mg/mL to about 10 mg/mL.

In any aspect of the disclosure, or embodiments thereof, the aqueous mixture of a) is at a temperature of from about 30° C. to about 80° C.

In any aspect of the disclosure, or embodiments thereof, the mixture containing the grain sample and the FAM-labeled starch substrate is incubated for between about 15 seconds and 5 minutes prior to b). In any aspect of the enclosure, or embodiments thereof, the mixture containing the grain sample and the FAM-labeled starch substrate is incubated for between 15 seconds and 1 minute prior to b).

In any aspect of the disclosure, or embodiments thereof, the separating of b) is carried out using filtration of centrifugation.

In any aspect of the disclosure, or embodiments thereof, c) involves quantifying the concentration of FAM in the soluble fraction using a fluorimeter. In any aspect of the disclosure, or embodiments thereof, b) involves quantifying the amount of FAM-labeled starch fragments in the soluble fraction using a lateral flow device (LFD) containing an anti-fluorescein antibody conjugated to a colloidal gold particle, a test line and a control line, where: i) the test line contains a polypeptide conjugated to fluorescein; and ii) the control line contains an antibody capable of binding to the anti-fluorescein antibody.

In any aspect of the disclosure, or embodiments thereof, the LFD contains a) a first portion containing a site for application of a liquid sample, a liquid-permeable medium, and the anti-fluorescein antibody conjugated to the colloidal gold particle. The LFD further contains b) a second portion in capillary communication with the first portion and containing a liquid-permeable medium containing, in order of distance from the first portion, the test line and the control line. The LFD also contains c) a third portion in capillary communication with the second portion and containing a liquid-permeable medium. In any aspect of the disclosure, or embodiments thereof, the liquid-permeable medium of a) contains polyester. In any aspect of the disclosure, or embodiments thereof, the liquid-permeable medium of b) contains nitrocellulose. In any aspect of the disclosure, or embodiments thereof, the liquid-permeable medium of c) contains cellulose.

In any aspect of the disclosure, or embodiments thereof, the third portion further contains a barcode for identification of the LFD.

In any aspect of the disclosure, or embodiments thereof, using the LFD involves contacting the lateral flow device with the soluble fraction for a period of from about 1 minute to about 5 minutes prior to reading the lateral flow device.

In any aspect of the disclosure, or embodiments thereof, the polypeptide of i) is bovine serum albumin (BSA).

In any aspect of the disclosure, or embodiments thereof, the method further involves predicting a Falling Numbers value for the grain sample based upon the amount of FAM-labeled starch fragments in the soluble fraction. In any aspect of the disclosure, or embodiments thereof, the Falling Numbers value is between 100 sec and 400 sec.

In any aspect of the disclosure, or embodiments thereof, the extract solution of c) is at a temperature of from about 30° C. to about 80° C. In any aspect of the disclosure, or embodiments thereof, the extract solution of a) is at a temperature of between about 65° C. and 75° C.

In any aspect of the disclosure, or embodiments thereof, the extract solution containing the FAM-labeled starch substrate is incubated for between about 0.5 minutes and 10 minutes prior to d).

In any aspect of the disclosure, or embodiments thereof, the extract solution comprising the FAM-labeled starch substrate is incubated for between 1 minute and 2 minutes prior to d).

In any aspect of the disclosure, or embodiments thereof, the method further involves filtering or centrifuging the extract solution after c) and prior to d).

In any aspect of the disclosure, the method does not involve a filtration step.

In any aspect of the disclosure, or embodiments thereof, d) involves quantifying the concentration of FAM in the extract solution using a fluorimeter.

In any aspect of the disclosure, or embodiments thereof, d) involves quantifying the amount of FAM-labeled starch fragments in the extract solution using a lateral flow device (LFD) containing an anti-fluorescein antibody conjugated to a colloidal gold particle, a test line and a control line, where: i) the test line contains a polypeptide conjugated to fluorescein; and ii) the control line contains an antibody capable of binding to the anti-fluorescein antibody.

In any aspect of the disclosure, or embodiments thereof, the grain sample contains ground barley, oats, rice, rye, spelt, teff, triticale, wheat, rice, sorghum, millet, maize, amaranth, buckwheat, chia, quinoa, or kaniwa. In any aspect of the disclosure, or embodiments thereof, where the grain sample contains or contains only a powder ground to a particle size so that the grain sample passes through a 12 to 40 mesh sieve with less than 5% retain by mass. In any aspect of the disclosure, or embodiments thereof, the grain sample contains or contains only a powder ground to a particle size so that the grain sample passes through a 20 mesh sieve with less than 5% retain by mass.

In any aspect of the disclosure, or embodiments thereof, the tablet contains between 40 mg and 160 mg of the FAM-labeled starch substrate.

In any aspect of the disclosure, or embodiments thereof, the volatile organic substrate is methanol.

In any aspect of the disclosure, or embodiments thereof, the first portion is between about 25 and 35 mm in length, the second portion is between about 20 and 30 mm in length, and the third portion is between about 35 and 45 mm in length. In any aspect of the disclosure, or embodiments thereof, the width of the lateral flow device is between about 1 and 5 mm.

In any aspect of the disclosure, or embodiments thereof, the liquid-permeable medium of a) contains polyester, the liquid-permeable medium of b) contains nitrocellulose, and the liquid-permeable medium of c) contains cellulose

In any aspect of the disclosure, or embodiments thereof, the method involves contacting the extract solution with the lateral flow device for less than 5 minutes. In any aspect of the disclosure, or embodiments thereof, the method is carried out in less than 15 minutes.

In any aspect of the disclosure, or embodiments thereof, the method is carried out in less than 10 minutes.

Compositions and articles defined by the invention were isolated or otherwise manufactured in connection with the examples provided below. Other features and advantages of the invention will be apparent from the detailed description, and from the claims.

In any aspect of the disclosure, or embodiments thereof, stopping the reaction comprises adding sodium tetraborate to the extract solution and/or raising the pH of the extract solution to greater than 8.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alpha-amylase polypeptide” is meant a protein having an amino acid sequence with at least 85% sequence identity to GenBank Accession No. AAA34259.1, which is provided below, or a fragment thereof capable of hydrolyzing alpha-bonds of alpha-linked polysaccharides, such as starch or glycogen.

>AAA34259.1 alpha-amylase [Triticum aestivum]
(SEQ ID NO: 1)
MGKHSATLCGLLVVVLCLASSLAQAQILFQGFNWESWKTQGGWYKFMQGKVEEIASTGATHVWL
PPPSQSVSPEGYLPGQLYNLNSKYGSGADLKSLIQAFRGKNISCVADIVINHRCADKKDGRGVY
CIFEGGTSDNRLDWGPDEICSDDTKYSNGRGHRDTGGGFDAAPDIDHLNPRVQRELSAWLNWLK
TDLGFDGWRLDFAKGYSAAMAKIYVDNSKPAFVVGELYDRDRQLLANWVRGVGGPATAFDFPTK
GVLQEAVQGDLGRMRGSDGKAPGMIGWMPEKTVTFIDNHDTGSTQRLWPFPSDKVMQGYAYILT
HPGIPCIFYDHVFDWKLKQEITALATVRSRNGIHPGSTLDILKAEGDLYVAKIGGKVITKIGSR
YNIGDNVIPSGFKIAAKGNNYCVWEKSGL.

By “alpha-amylase polynucleotide” is meant a polynucleotide encoding an alpha-amylase polypeptide.

By “alteration” is meant an increase or decrease in an analyte.

By “analog” is meant a molecule that is not identical to another molecule but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “analyte” is meant any agent under investigation using an analytical method. In embodiments, an analyte is a water-soluble starch fragment covalently bound by a detectable moiety (e.g., fluorescein).

By “analyte-binding conjugate” is meant a detectable moiety that binds a compound under investigation. In embodiments, the analyte-binding conjugate contains an antibody capable of binding to fluorescein that is bound to a colloidal gold particle.

By “anti-fluorescein antibody” is meant an antibody that specifically binds fluorescein. Non-limiting examples of anti-fluorescein antibodies include 1F11 (Jackson Immunoresearch Laboratories), ab19491 (abcam), and Anti-Fluorescein (FITC) Antibody, and clone 5D6.2 (SigmaAldrich). In some embodiments, the antibody is a monoclonal antibody.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “capillary communication” is meant facilitating the flow of a liquid between liquid-permeable materials.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes (e.g., fluorescein), electron-dense reagents, enzymes (for example, as commonly used in an ELISA), biotin, digoxigenin, or haptens.

By “florescein (FAM)” is meant a compound with the structure

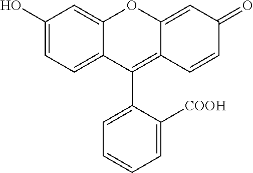 [SMILES: O=C(O)c1ccccc1-c1c2ccc(=O)cc-2oc2cc(O)ccc12]

corresponding to IUPAC name 3′,6′-dihydroxyspiro[isobenzofuran-1 (3H),9′-[9H]xanthen]-3-one and CAS No. 2321 Jul. 5, as well as salts thereof. In embodiments, the salt is a sodium salt.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increase” is meant to alter positively relative to a reference. An increase may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “insoluble” is meant a molecule or particle that may be removed from a liquid using methods such as filtration or centrifugation. In embodiments, the liquid is water.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “lateral flow device” is meant a device suitable for use in detecting an analyte in a sample and that relies on the flow of a liquid via capillary action, wicking, or wetting a liquid-permeable media present in the device.

By “liquid-permeable material” is meant a material susceptible to wetting, wicking, or transport of a liquid by capillary action.

By “natural starch” is meant a starch isolated from an organism capable of producing starch. In embodiments, the natural starch is corn starch. In some embodiments, the natural starch is isolated from a grain.

By “non-detectable” is meant an analyte that cannot be detected using the method employed by the laboratory.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively relative to a reference. A reduction may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “specifically binds” is meant binds a target analyte without substantially binding to other non-target compounds present in the sample.

By “starch” is meant a polymeric carbohydrate containing numerous glucose units joined by glycosidic bonds. Starch is produced by green plants for energy storage and is contained in large amounts in staple foods, such as wheat, potatoes, maize (corn), rice, and cassava. Non-limiting examples of foods containing starch include cereal or pseudocereal grains, such as barley, oats, rice, rye, spelt, teff, triticale, wheat, rice, sorghum, millet, maize, amaranth, buckwheat, chia, quinoa, or kaniwa samples, or products thereof.

By “suspension” or “suspending” is meant a mixture or preparing a mixture in which solid particles within the suspension do not dissolve but become distributed in a solvent and may be separated from the solvent. In embodiments, the separation involves filtration, sedimentation, and/or centrifugation. In some embodiments, the solvent is water.

By “wick” is meant sorb a liquid.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic diagram describing the design of a lateral flow device of the disclosure.

FIG. 2 provides a schematic diagram showing a method for using a lateral device (LFD) of the disclosure. In FIG. 2, “FAM” indicates a fluorescein molecule (e.g., 6-amino fluorescein).

FIG. 3 provides a schematic diagram describing the design and operation of a lateral flow device (LFD) of the disclosure. In FIG. 3, the term “FAM” indicates a fluorescein molecule (e.g., 6-amino fluorescein), “Ab” indicates a monoclonal antibody, “TL” indicates a test line, “CL” indicates a control line, and “FAM-BSA” indicates bovine serum albumin covalently linked to a FAM. In FIG. 3, “Positive LFD” indicates a lateral flow device providing a positive readout indicating that a sample contains digested FAM product, and “Negative LFD” indicates a negative readout indicating that a sample does not contain digested FAM product.

FIG. 4 provides a schematic diagram depicting lateral flow device readouts obtained using the lateral flow device of FIGS. 1 and 3 and corresponding to a positive (+) result indicating the presence of a digested FAM product in a sample, and to a negative (−) result indicating the absence of a digested FAM product in a sample.

FIG. 5 provides a schematic diagram showing an embodiment of a fluorescein (FAM)-labeled starch substrate of the disclosure.

FIG. 6 provides a schematic diagram showing an embodiment of bovine serum albumin (BSA), such as thiolated BSA, covalently linked to a fluorescein (FAM) (e.g., 6-amino fluorescein).

FIG. 7 provides a schematic diagram showing a method for detection of alpha-amylase activity in a sample using a FAM-labeled starch substrate (“FAM-substrate”) of the disclosure.

FIG. 8 provides a plot showing increase in fluorescence over time in an aqueous solution containing a FAM-labeled starch substrate of the disclosure and a winter wheat sample having one of the indicated pre-measured Falling Number assignments (i.e., 150 sec, 260 sec, or 400 sec). In FIG. 8, “autoclaved” indicates a negative control containing an autoclaved winter wheat sample. In FIG. 8, “RFUs” indicate relative fluorescence units, and “ELX substrate” indicates a FAM-labeled starch substrate of the disclosure.

FIG. 9 provides a plot showing increase in absorbance at 620 nm (i.e., optical density (OD)) over time in an aqueous solution containing the commercially available synthetic biochemical substrate Phadebas™ and a winter wheat sample having one of the indicated pre-measured Falling Number assignments (i.e., 150 sec, 260 sec, or 400 sec). In FIG. 9, “autoclaved” indicates a negative control containing an autoclaved winter wheat sample.

FIG. 10 provides a plot showing increase in absorbance at 400 nm (i.e., optical density (OD)) over time in a commercially available Megazyme assay. The Megazyme assay was conducted using an alpha-amylase SD assay kit (Product Code: K-AMYLSD) available from Megazyme, which contained ethylidene-end blocked p-nitrophenyl maltoheptaoside as the substrate used for detecting alpha-amylase activity. The Megazyme assay was used to measure alpha-amylase activity in a winter wheat sample having one of the indicated pre-measured Falling Number assignments (i.e., 150 sec, 260 sec, or 400 sec). In FIG. 10, “autoclaved” indicates a negative control containing an autoclaved winter wheat sample.

FIG. 11 provides a plot comparing increase in fluorescence or optical density over time corresponding to the winter wheat samples of FIGS. 8-10 (the ELX, Phadebas™, and Megazyme curves of FIG. 11, respectively) assigned a Falling Number of 150 seconds. The fluorescence or optical density measurements were normalized to endpoint fluorescence measurements.

FIGS. 12A and 12B provide plots showing the consistency in measurements of alpha-amylase activity between assays using different amounts of a FAM-labeled starch substrate of the disclosure (i.e., 3 tablets, 4 tablets, or 5 tablets). Each tablet contained about 2 mg of the FAM-labeled starch substrate. FIG. 12A provides a plot showing increase in fluorescence over time in an aqueous solution containing the indicated amount of the FAM-labeled starch substrate of the disclosure and a winter wheat sample having a pre-measured Falling Number assignments of 150 sec. In FIG. 12A, the term “RFUs” indicates relative fluorescence units, and the term “ELX substrate” indicates the FAM-labeled starch substrate of the disclosure. FIG. 12B provides a plot depicting the data of FIG. 12A normalized to endpoint fluorescence measurements.

FIG. 13 provides microscopic images. The left panel of FIG. 13 provides a representative image of a starch sample used to prepare the FAM-labeled starch substrate shown in the right panel of FIG. 13. In FIG. 13, the term “um” indicates micrometers.

FIG. 14 provides incandescent and fluorescence microscopy images of a FAM-labeled starch substrate of the disclosure. The left panel of FIG. 14 provides an incandescent microscopy (i.e., microscopy carried out using white light) image of a FAM-labeled starch substrate of the disclosure, and the right panel of FIG. 14 provides the fluorescence microscopy image corresponding to the image of the left panel of FIG. 14. In FIG. 14, the term “um” indicates micrometers.

FIG. 15 provides a schematic diagram showing an embodiment of a method of the disclosure for preparing a FAM-labeled starch substrate.

FIG. 16 provides a plot showing a correlation between endpoint fluorescence (i.e., relative fluorescence units (RFU)) measured using FAM-labeled starch substrates of the disclosure according to the methods of the disclosure and Falling Number Assignment in seconds (secs) for winter wheat samples.

FIGS. 17A and 17B provide plots showing a correlation between test line (TL) fluorescence measurements and Falling Number assignments in seconds (secs) for winter wheat samples. The test line fluorescence measurements were obtained by using lateral flow devices of the disclosure to measure levels of soluble FAM-labeled starch molecules in an aqueous solution containing a FAM-labeled starch substrate of the disclosure and a winter wheat sample with a pre-measured Falling Number (FN) Assignment in seconds (secs) as indicated along the x-axis. In FIGS. 17A and 17B, the term “Poly.” indicates polynomial curve fitting.

FIG. 18 provides a plot showing a correlation between the Signal Intensity (SI) and Falling Number assignments in seconds for winter wheat samples. The Signal Intensity measurements were measured using the lateral flow devices of the disclosure. Signal Intensity was calculated using the following formula: SI=(TL/(TL+CL))*100, where “TL” indicates the gold signal intensity measured at the Test Line and “CL” indicates the gold signal intensity measured at the Control Line.

### DETAILED DESCRIPTION OF THE INVENTION

The disclosure features lateral flow devices (LFDs) and insoluble fluorescence (FAM)-labeled starch substrates, compositions comprising the same, and methods for use thereof in the measurement of alpha amylase activity in a sample.

The disclosure is based, at least in part, upon the discovery of an insoluble FAM-labeled starch substrate suitable for use in methods for detecting alpha amylase activity in a sample and of LFDs for use in said methods. In particular, it was found, as described further in the Examples provided herein, that the methods can be used as an alternative to the Falling Numbers method and as a reliable method for predicting a Falling Numbers assignment for a sample. It can be advantageous for the FAM-labeled starch substrate to be insoluble in water because, among other things, such insolubility allows for the ready removal of intact FAM-labeled starch substrate from a reaction mixture.

### Alpha-Amylase Activity

Alpha-amylase (α-Amylase) is an enzyme (EC 3.2.1.1; systematic name 4-α-D-glucan glucanohydrolase) that hydrolyses alpha bonds of large, alpha-linked polysaccharides, such as starch and glycogen, yielding shorter chains thereof, dextrins, and maltose: Endohydrolysis of (1→4)-α-D-glucosidic linkages in polysaccharides containing three or more (1→4)-α-linked D-glucose units.

High levels of alpha-amylase activity in a grain or grain product are indicative of low quality thereof. For example, too much alpha amylase activity in a flour will result in too much sugar being present and not enough starch, which affects many aspects of the breadmaking process, such as dough processing, baking, slicing and the overall appearance of the bread. The flour absorbs less water and the dough is soft and sticky, which makes dough handling difficult. During the baking process, large, open holes are formed and sticky crumb, which accumulates on slicer blades. Also, the large amount of sugar present causes the crust to become too dark and appear burnt. The result is therefore that bread loaves are deformed, difficult to slice and have too dark crusts, all of which are unattractive to customers.

Too much alpha-amylase in cereals is typically caused by either pre-harvest sprouting, which is caused by poor weather conditions before harvest (usually heavy rainfall) causing the grain to sprout too early, or by “late maturity alpha-amylase”, which is a genetic defect. Some wheat cultivars tend to have abnormally high levels of alpha-amylase, which may be triggered by extreme temperatures (very cold or very hot) or cool conditions that carry on for a long time. Genetics can further influence alpha-amylase activity.

Since its introduction in the early 1960s, the Falling Numbers test has become a world standard in the grain and flour milling industries for measuring alpha-amylase activity in wheat, durum wheat, triticale, rye and barley, as well as milled products made from these grains. The Falling Numbers is used to assign a Falling Number (FN) value (also referred to as Hagberg number of Hagberg-Perten number) to a sample. The international standard for measurement of Falling Numbers corresponds to ICC 107/1, ISO 3093-2004, AACC 56-81B. The Falling Number of a sample is very closely related to the concentration of alpha amylase in a sample.

The Falling Number method requires an apparatus which follows the international standards (ICC 107/1, ISO 3093-2004, AACC 56-81B). Such an apparatus consists of a water bath, a test tube, a stirring rod, and a stirring device. The test was performed manually when first employed, test instrumentation today is mostly automated.

To analyze a grain sample, it first needs to be ground to a powder; a flour sample can be analyzed as is. The sample is put into the test tube; distilled water is added, and the tube is then shaken vigorously to achieve a homogeneous mix. The tube is then placed in the boiling water bath, and the operator begins to stir the sample. Simultaneously the starch begins to gelatinize and the slurry becomes more viscous. The mixing ensures the gelatinization is homogeneous in the slurry. An additional effect of the high temperature is that the alpha-amylase enzyme contained in the grain begins to break the starch down into glucose and maltose, thereby reducing the viscosity of the slurry. The amount of starch break-down is directly proportionate to the alpha-amylase activity, meaning that the higher the activity of the alpha-amylase, the lower the viscosity will be.

After 60 seconds of mixing, the stirrer is dropped from the top of the test tube, and the operator measures the time it takes for the stirrer to reach the bottom. That time, measured in seconds, is the Falling Number. When the stirrer is dropped, its speed and thus the time it takes for it to reach the bottom, will be determined by the viscosity of the slurry. In other words, the more sprouted the grain was, the higher the alpha-amylase activity will be. The higher the alpha-amylase activity the lower the viscosity of the slurry. The lower the viscosity of the slurry the faster the stirrer will fall to the bottom. That is why more sprouted grain results in a lower Falling Number as Falling Number is the time it takes the stirrer to fall to the bottom. The FN value has an inverse relationship with the alpha-amylase activity, meaning the higher the alpha-amylase activity the lower the FN value, and vice versa.

In general, values below 300 seconds are indicative of poor quality for milling and baking purposes. Sprout damaged grain can affect mixability, crumb strength, sliceablity, and volume of bread prepared using flour produced from the grain. Wheat that does not meet the minimum falling number standard is unsuitable for milling and is usually sold to the livestock feed market.

### Labeled Substrate

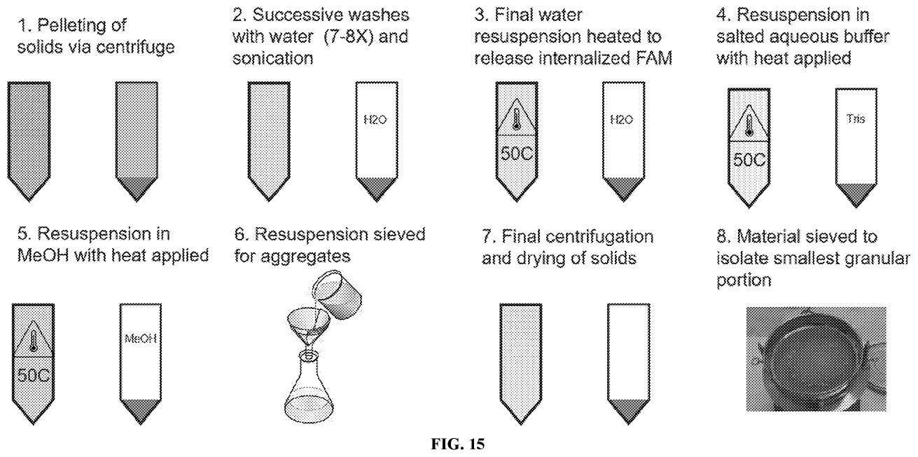

The present disclosure provides a water-insoluble starch labeled with a detectable label (e.g., fluorescein (FAM)) suitable for use as a substrate in assays for the detection of alpha-amylase activity in a sample. In embodiments, the substrate is a FAM-labeled starch substrate. The FAM-labeled starch substrate may be produced according to the methods provided herein, such as by the method shown in FIG. 15.

Preparation of the FAM-labeled starch substrate involves covalently linking a detectable label, such as fluorescein, to a starch polymer followed by washing and drying steps. Fluorescein may be covalently linked to a starch by reacting the starch in a first reaction mixture with carbonyldiimidazole (CDI) to activate accessible hydroxyl groups, reacting fluorescein in a second reaction mixture with adipic acid dihydrazine (AAD) in the presence of 1-Ethyl-3-(3-dimethylaminopropyl) carbodiimide (EDC) so that the carboxyl group of the fluorescein becomes covalently linked to AAD, and then combining the two reaction mixtures to allow the AAD linked to the fluorescein to react with the activated starch hydroxyl groups to thereby covalently link fluorescein to the starch particles. Following these reaction steps, the resulting FAM-labeled starch substrate is then subjected to one or more of the following wash steps to remove unconjugated components from the reaction.

In various embodiments, the starch particles are washed between 5 and 10 times by centrifuging the mixture containing the FAM-labeled starch substrate, removing the resulting supernatant, and subsequently resuspending the remaining pellet in purified water, such as MilliQ® water, distilled-deionized water, distilled water, or the like. In embodiments, the starch particles are washed until the supernatant is substantially free of color (i.e., a practitioner is unable to easily discern any color without the aid of any special equipment, such as a spectrophotometer or fluorimeter). The final washing step in purified water or any number of the washing steps in the purified water may include heating of the FAM-labeled starch substrate suspended in the purified water to a temperature between 30° C. and 100° C., 40° C. and 80° C., or 45° C. and 55° C. (e.g., at 50° C.) and incubating the mixture for about or at least about 1 min, 2 min, 3 min, 4 min, 5 min, 10 min, 15 min, 20 min, or 30 min prior to separating the FAM-labeled starch substrate from the purified water (e.g., by centrifugation or filtration). In embodiments, the incubation is less than about 2 min, 3 min, 4 min, 5 min, 10 min, 15 min, 20 min, or 30 min.

It is contemplated that the washing steps may use any method available in the art for separation of the FAM-labeled starch substrate from the wash solution (e.g., centrifugation or filtration). It is further contemplated that, in various embodiments, resuspension of the FAM-labeled starch substrate may involve the use of sonication.

In some embodiments, following the washing steps in purified water, the FAM-labeled starch substrate is washed one or more times (e.g., 1, 2, 3, 4, 5, 6, 7, 8, 9, or 10 times) by resuspending the FAM-labeled starch substrate in an aqueous buffered solution containing salt ions (e.g., Ca²⁺, Na⁺) followed by separation of the FAM-labeled starch substrate from the aqueous buffered solution containing the salt ions. In embodiments, the aqueous buffered solution contains a buffer containing primary amines (e.g., a Tris-buffer), which may assist in blocking any unreacted activated hydroxyl groups present in the FAM-labeled starch substrate. In some embodiments, the aqueous buffered solution contains Ca²⁺ ions or CaCl₂) at a concentration of about or at least about 2 mM, 4 mM, 6 mM, 8 mM, 10 mM, 15 mM, 20 mM, 25 mM, 30 mM, 35 mM, 40 mM, 45 mM, or 50 mM. In some embodiments, the aqueous buffered solution contains Ca²⁺ ions or CaCl₂) at a concentration of less than about 2 mM, 4 mM, 6 mM, 8 mM, 10 mM, 15 mM, 20 mM, 25 mM, 30 mM, 35 mM, 40 mM, 45 mM, or 50 mM. In embodiments, the aqueous buffered solution contains Ca²⁺ ions or CaCl₂) at a concentration of between about 15 mM and 30 mM (e.g., 20 mM). In embodiments, the FAM-labeled starch substrate resuspended in the aqueous buffered solution containing salt ions is incubated at a temperature between 30° C. and 100° C., 40° C. and 80° C., or 45° C. and 55° C. (e.g., at 50° C.) for about or at least about 1 min, 2 min, 3 min, 4 min, 5 min, 10 min, 15 min, 20 min, or 30 min prior to separation of the FAM-labeled starch substrate from the aqueous buffered solution containing the salt ions. In embodiments, the incubation is less than about 2 min, 3 min, 4 min, 5 min, 10 min, 15 min, 20 min, or 30 min. In embodiments, the buffer solution has a pH of about or at least about 5, 5.5, 6, 6.5, 7, 7.5, 8, 8.5, or 9. In embodiments, the buffer solution has a pH of less than about 5.5, 6, 6.5, 7, 7.5, 8, 8.5, or 9.

In embodiments, a final washing step or set of washing steps (e.g., 1, 2, 3, 4, 5, 6, 7, 8, 9, or 10 washing steps) used in preparation of the FAM-labeled starch substrate involves resuspending the FAM-labeled starch substrate in a volatile organic liquid (e.g., a polar liquid, such as methanol) followed by separation of the organic alcohol from the FAM-labeled starch substrate (e.g., through centrifugation or filtration). In embodiments the volatile organic liquid contains little-to-no water (e.g., less than 5%, 4%, 3%, 2%, 1%, 0.1%, 0.01%, 0.001%). In embodiments, the FAM-labeled starch substrate resuspended in organic alcohol is incubated at a temperature between 30° C. and 100° C., 40° C. and 80° C., or 45° C. and 55° C. (e.g., at 50° C.) for about or at least about 1 min, 2 min, 3 min, 4 min, 5 min, 10 min, 15 min, 20 min, or 30 min prior to separation of the FAM-labeled starch substrate from the volatile organic liquid (e.g., methanol). In embodiments, the incubation is less than about 2 min, 3 min, 4 min, 5 min, 10 min, 15 min, 20 min, or 30 min.

In embodiments, the final washing step or set of washing steps is followed by filtering the mixture containing the volatile organic solvent and the FAM-labeled starch substrate to remove aggregates containing the FAM-labeled starch substrate. For example, such filtration may involve passing the mixture through multiple layers of cheesecloth.

Following the washing steps, the FAM-labeled starch substrate is dried. The drying can be carried out at a temperature of about or at least about 10° C., 15° C., 20° C., 25° C., 30° C., 35° C., 40° C., 45° C., 50° C., 55° C., 60° C., or higher. In embodiments, the temperature is less than about 15° C., 20° C., 25° C., 30° C., 35° C., 40° C., 45° C., 50° C., 55° C., or 60° C. The drying can have a duration of about or at least about 1 hour, 2 hours, 3 hours, 4 hours, 5 hours, 6 hours, 12 hours, 24 hours, 2 days, 3 days, or more. The drying can have a duration of less than about 1 hour, 2 hours, 3 hours, 4 hours, 5 hours, 6 hours, 12 hours, 24 hours, 2 days, or 3 days.

Once the FAM-labeled starch substrate has been dried, the FAM-labeled starch substrate may be sieved to isolate a portion of the FAM-labeled starch substrate having a desired particle size. In some embodiments, the average particle size of the sieved FAM-labeled starch substrate is about or at least about 5 μm, 10 μm, 15 μm, 20 μm, 25 μm, 30 μm, 35 μm, 40 μm, 45 μm, or 50 μm and/or no greater than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, or 250 μm. In some embodiments, the average particle size of the sieved FAM-labeled starch substrate is less than about 5 μm, 10 μm, 15 μm, 20 μm, 25 μm, 30 μm, 35 μm, 40 μm, 45 μm, or 50 μm and/or no greater than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, or 250 μm. In some embodiments, the FAM-labeled starch substrate is sieved using an 80-mesh sieve.

In various embodiments, preparation of the FAM-labeled starch substrate involves preparing tablets containing the FAM-labeled starch substrate. The tablets can be prepared using methods available in the art. For example, the tablets can be prepared by combining the FAM-labeled starch substrate with microcrystalline cellulose (e.g., MCC102) and using the resulting mixture to prepare tablets using a tableting device, such as an LFA tablet press. In embodiments, the resulting tablets contain about or at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg of the FAM-labeled starch substrate. In embodiments, the resulting tablets contain no more than about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg of the FAM-labeled starch substrate. The tablets may be any of a diversity of different shapes and sizes suitable for use in a particular application. In some embodiments, the tablets are cylindrical in shape and have a diameter of about, or at least about, 1 mm, 2 mm, 3 mm, 4 mm, 5 mm, 6 mm, 7 mm, 8 mm, 9 mm, or 10 mm (e.g., 6 mm), and a height/thickness of about or at least about 0.5 mm, 1 mm, 1.5 mm, 2 mm, 2.5 mm, 3 mm, 3.5 mm, 4 mm, 4.5 mm, or 5 mm (e.g., 3.2 mm). In some embodiments, the tablets are cylindrical in shape and have a diameter of less than about 1 mm, 2 mm, 3 mm, 4 mm, 5 mm, 6 mm, 7 mm, 8 mm, 9 mm, or 10 mm, and a height/thickness of less than about 0.5 mm, 1 mm, 1.5 mm, 2 mm, 2.5 mm, 3 mm, 3.5 mm, 4 mm, 4.5 mm, or 5 mm.

### Lateral Flow Device (LFD)

The present invention features compositions and methods that are useful for the detection of an analyte (e.g., a fragment of a starch molecule covalently coupled to fluorescein) in a sample. In particular, the invention provides a lateral flow device (LFD), that comprises a liquid-permeable media that provides for the flow of a liquid sample through the device. LFDs of the invention can be used for the detection of water-soluble starch fragment covalently bound to a detectable label as an analyte of interest. The LFD described herein is particularly suitable for the detection of an analyte of interest using an antibody that specifically binds the analyte. The LFD and methods of the invention are generally useful for the detection of virtually any analyte, antigen, or hapten bound to a detectable label (e.g., a fluorophore, such as fluorescein).

As described further below, the disclosure provides methods of using a LFD of the disclosure for the detection of an analyte (e.g., an antigen, such as fluorescein) in a sample. In one example, the assay is conducted by placing the leading edge (first portion) of a lateral flow device in contact with a liquid sample. In another example, the sample is brought into contact with the device by applying a liquid sample to the first portion of the lateral flow device in a drop-wise fashion.

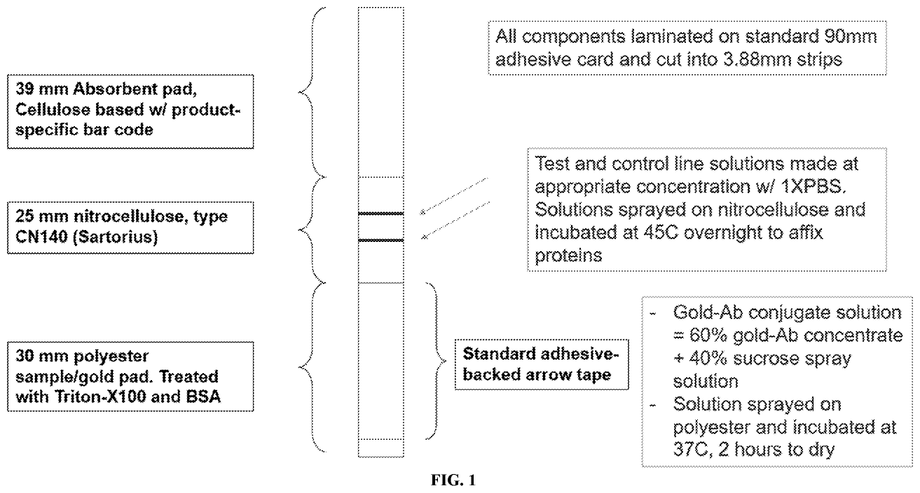

The lateral flow device (LFD) can take any form desired that provides for the flow of a liquid sample from the point of contact with the sample past a test and/or control line. In general, the LFD of the present invention includes an interior flow pathway that includes one or more liquid-permeable materials. A schematic diagram showing an exemplary LFD is provided at FIGS. 1A and 1B in WO 2007/092302, the disclosure of which is incorporated herein by reference in its entirety for all purposes. In a first portion, the device contains a material (e.g., polyester) that provides for the flow of the liquid sample therethrough and includes a site for the application of a liquid sample. This first portion of the device also includes an analyte-binding conjugate (e.g., an anti-fluorophore antibody conjugated to a gold particle), such as an antibody that specifically binds an analyte of interest. The analyte binding conjugate typically binds the analyte to form a complex. Complex formation (e.g., formation of an analyte/antibody-conjugate complex) may occur at any point in the interior flow pathway after the analyte contacts the analyte-binding conjugate. For example, complex formation may occur or continue as the sample flows from the first portion to the second portion of the device. In various embodiments, it can be advantageous to treat the first portion so that it has reduced binding to polypeptides (e.g., treating the first portion with a surfactant, such as Triton-X100, and a blocking polypeptide, such as bovine serum albumin).

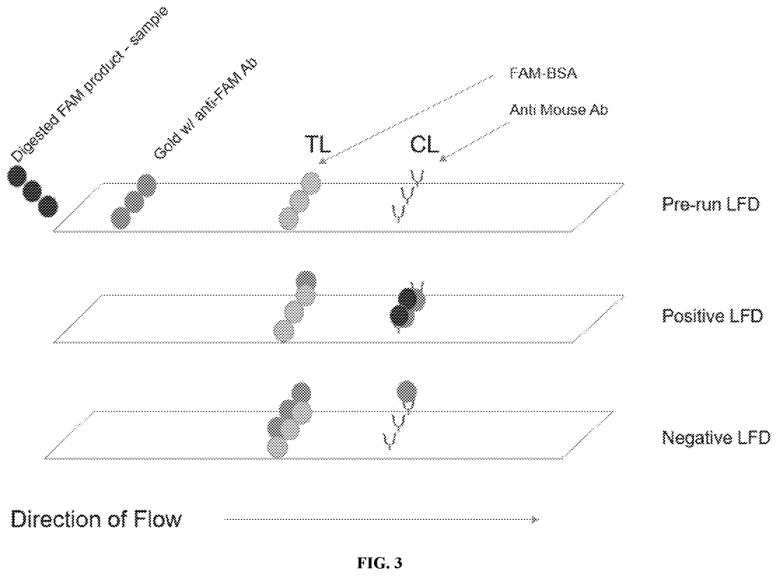

In a second portion downstream of the first portion, the LFD contains a material (e.g., nitrocellulose) providing for the flow the liquid sample therethrough and containing a test line. The test line contains a molecule that binds analyte-binding conjugates that are not bound to an analyte. In embodiments, the analyte-binding conjugate binds a fluorophore and the test line (TL) contains the fluorophore (e.g., in the form of fluorescein conjugated to bovine serum albumin) to which the analyte-binding conjugate binds so that analyte-binding conjugates bound by an analyte will flow past the test line without binding to the fluorophore, and analyte-binding conjugates that are not bound by an analyte will not flow past the test line and will bind to the fluorophore (see, e.g., FIG. 3). Downstream of the test line and within the material providing for the follow of the liquid sample therethrough is a control line, which contains an antibody capable of binding the analyte-binding conjugates so that any antibody-binding conjugates that flow past the test line will fail to flow past the following downstream control line (CL). In an exemplary embodiment, the antibody-binding conjugate is a monoclonal mouse anti-fluorescein antibody conjugated to a gold particle and the antibody capable of binding the analyte-binding conjugate is an anti-mouse monoclonal or polyclonal antibody.

The device may also include in a third portion containing wicking pad that contains sorbent material (e.g., a cellulose based pad) capable of absorbing or adsorbing excess liquid present in the liquid sample. The third portion can be labeled with a product-specific bar code. In one embodiment, the LFD contains a liquid-permeable material defining the following portions in capillary communication:
a) a first portion that is the site for application of a liquid sample, comprising a liquid-permeable medium (e.g., polyester) and an anti-analyte antibody conjugate (e.g., a gold-antibody conjugate containing an antibody capable of binding fluorescein, such as the monoclonal antibody 1F11 available from Jackson Immunoresearch Laboratories), where the first portion may be between about 5 mm and 60 mm in length; for example, the length of the first portion is equal to any value between about 5 and 60 (e.g., 5, 10, 15, 20, 25, 30, 35, 40, 45, 50, 55, 60 mm in length), and where the first portion may be treated with a surfactant (e.g., Triton-X100) and/or a blocking polypeptide (e.g., bovine serum albumin (BSA));
c) a second portion containing a liquid-permeable medium (e.g., nitrocellulose) that is the site for detecting the binding of the anti-analyte antibody conjugate unbound by an analyte (e.g., water-soluble starch fragment covalently bound to fluorescein) at a test line (e.g., by fluorescein covalently bound to BSA) and the binding of the anti-analyte antibody conjugate bound by an analyte at a control line (e.g., by an antibody capable of binding the antibody capable of binding fluorescein, such as an anti-mouse polyclonal antibody), the second portion contains a liquid-permeable medium having the analyte (e.g., fluorescein) fixed to the medium at the test line, and having an antibody that binds the anti-analyte antibody conjugate (e.g., a polyclonal anti-mouse antibody) present at a control line, where the second portion is between about 10 and 50 mm in length; for example, is any value between about 10 and 50 (e.g., 10, 15, 20, 25, 30, 35, 40, 45, 50), and, in some embodiments, the second portion overlaps the first portion by at least 1 2, 3, 4, or 5 mm; and
d) a third portion containing a liquid-permeable medium containing a wicking pad that contains sorbent material (e.g., a cellulose based pad), where the second portion is between about 15 and 80 mm in length; for example, is any value between about 15 and 80 (e.g., 15, 20, 25, 30, 35, 40, 45, 50, 55, 60, 65, 70, 75, 80), where in some embodiments the third portion contains a product-specific barcode.

In one embodiment, the third portion overlaps the second portion by at least 1, 2, 3, 4, or 5 mm. In various embodiments, the liquid-permeable media of the LFD are secured to a common solid support via an adhesive. In embodiments, the support is a card made of a suitable material.

In general, the interior flow path of the lateral flow device is between 1 mm and 10 mm in width (e.g., about 3.88 mm); for example, the width of a LFD is any value between 1 and 10. In one embodiment the width of the strip is 3.8 mm. The design and dimensions of an exemplary lateral flow are shown in FIG. 1. In some embodiments, the width of the LFD is about or at least about 1 mm, 2 mm, 3 mm, 4 mm, 5 mm, 6 mm, 7 mm, 8 mm, 9 mm or 10 mm. In some embodiments, the width of the LFD is less than about 2 mm, 3 mm, 4 mm, 5 mm, 6 mm, 7 mm, 8 mm, 9 mm or 10 mm.

### Antibodies

As described herein, the lateral flow device includes a conjugate that binds an analyte. In one approach, the conjugate is an antibody capable of binding an antigen capable of generating an immune response (e.g., fluorescein), either alone or when conjugated to another compound, as in the case of a hapten. Any antibody, antibody conjugate, or fragment thereof that binds an antigen or analyte of interest may be used in the present invention. Such antibodies or antibody conjugates are present within the interior flow path of the lateral flow device. Suitable antibodies include, but are not limited to, polyclonal antibodies, monoclonal antibodies, or fragments thereof. As used herein, the term “antibody” means not only intact immunoglobulin molecules but also the well-known active fragments F(ab′) 2 and Fab. In some embodiments, the selected antibody binds an antigen of interest with high affinity. In some embodiments, the antibody binds a detectable label, such as a fluorophore like fluorescein. Exemplary embodiments of antibodies capable of binding fluorescein that are suitable for use in lateral flow devices and methods of the disclosure include but are not limited to: 1F11 (Jackson Immunoresearch Laboratories), ab19491 (abcam), and Anti-Fluorescein (FITC) Antibody, and clone 5D6.2 (SigmaAldrich).

Antibodies having a desired binding characteristic may be identified using methods known to the skilled artisan. One method of obtaining antibodies is to immunize a suitable host animal (e.g., a mouse or rabbit) with an immunogen (e.g., a molecule containing fluorescein) and to follow standard procedures for polyclonal or monoclonal antibody production. The desired antibodies, cells expressing the same, and/or polynucleotides encoding the same are then isolated from the host. The antibodies can then be purified from cells expressing the antibodies. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

In one approach, antibodies are produced from hybridoma cells engineered to express a desired antibody. Methods of making hybridomas are well known in the art. The hybridoma cells can be cultured in a suitable medium and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

To detect the antibody/antigen complex within the LFD, a detector reagent, or conjugate, must be coupled to the antibody or antigen (e.g., colloidal gold). Exemplary conjugates include colored reagents, fluorescent compounds, enzymes, and radioactive isotopes. Colored or fluorescent compounds include gold particles (e.g., colloidal gold), colored or fluorescent latex particles, polystyrene beads, and dyes, such as fluorescein isothiocyanate, BODIPY FL, Oregon Green, Alexa Fluor 488, phycoerythrin and phycocyanin. Colloidal metals, metal sols and other types of colored particles useful as marker substances in immunoassay procedures are described, for example, in U.S. Pat. No. 4,313,734, the disclosure of which is incorporated herein by reference in its entirety for all purposes. Antibody conjugates are widely available, for example, from a variety of commercial sources (e.g., Molecular Probes (e.g., Zenon® labeling technology), Nanoprobes (e.g., Nanogold® Gold-Antibody Conjugates).

Enzymes that may be coupled to an antibody include peroxidases (such as horseradish peroxidase), phosphatases (such as acid or alkaline phosphatase), β-galactosidase, urease, glucose oxidase, carbonic anhydrase, acetylcholinesterase, glucoamylase, lysozyme, malate dehydrogenase, glucose-6-phosphate dehydrogenase, β-glucosidase, proteases, pyruvate decarboxylase, esterases, luciferase, or any other enzyme known to the skilled artisan. Enzymes are not in themselves detectable but must be combined with a substrate to catalyze a reaction the end product of which is detectable.

Antibodies, antibody conjugates, protein-antigen conjugates, and protein-hapten conjugates may be fixed within the interior flow path using methods known to the skilled artisan. Protein immobilization protocols are known to the skilled artisan. See, for example, Laboratory Techniques in Biochemistry and Molecular Biology, Tijssen, Vol. 15, Practice and Theory of Enzyme Immunoassays, Chapter 13, The Immobilization of Immunoreactants on Solid Phases, pp. 297-328, and the references cited therein. In one approach, an antibody is immobilized directly on a solid support by physical adsorption or is bound covalently or through bridging molecules such as protein A, polylysine or to a solid support.

### Interior Flow Path

The LFD comprises an interior flow path that facilitates the flow of a liquid sample through the device. This interior flow path contains one or more liquid-permeable materials or membranes composed of any relatively inert material or a combination of materials suitable for transporting a liquid (e.g., glass fibers, polyester, nitrocellulose, fibers of cellulose or derivatives thereof, such as nitrocellulose, non-cellulose hydrocarbon materials, ceramics) from the contact site past the test and/or control sites and, optionally, into a reservoir. Suitable materials for use in the interior flow path are wettable and exhibit low non-specific binding. Materials having increased sorptivity promote the flow of liquid. Different materials having different absorption characteristics or sorptivities may be used in various portions of the flow path. If desired, the materials to be used are screened for optimal pore size and density in order to facilitate the controlled distribution of an antibody within a membrane, to optimize reaction kinetics, or to optimize the sensitivity, discriminatory ability, or signal-to-noise ratio of the device.

### Solid Supports

In various embodiments, the LFD includes an interior flow pathway fixed to a solid support. The physical shape of the solid support is not limiting, although some shapes may be more convenient than others for a particular application. Accordingly, the solid support may be in the shape of a paper strip, dipstick, membrane (e.g., a nylon membrane or a cellulose filter), a plate (e.g., a microtiter plate) or solid particles (e.g., latex beads). The solid support may be made of any suitable material, including but not limited to a plastic (e.g., polyethylene, polypropylene, polystyrene, latex, polyvinylchloride, polyurethane, polyacrylamide, polyvinylalcohol, nylon, polyvinyl acetate, or any suitable copolymers thereof), cellulose (e.g. various types of paper, such as nitrocellulose paper and the like), a silicon polymer (e.g. siloxane), a polysaccharide (e.g. agarose or dextran), or an ion exchange resin (e.g. conventional anion or cation exchange resins).

### Sorbent Reservoir

The LFD optionally includes a third portion that forms a reservoir of adsorbent or absorbent material. This reservoir sorbs excess liquid as it flows through the LFD. For some applications, such as where the concentration of antigen (e.g., water-soluble fluorescein bound to a starch fragment) in a sample is particularly low, it may be desirable to apply large volumes of a liquid sample to the LFD. In such cases, the presence of the adsorbent material may enhance the sensitivity of antigen detection. Optionally, the region of the flow path in the test cell defining the test and control sites is restricted in cross-sectional area relative to other regions of the flow path. This feature produces a “bottleneck” effect wherein the antigen in the entire volume of adsorbed sample must pass through an area of restricted flow immediately above the test site. This “bottleneck” may facilitate sandwich formation. Suitable sorbent materials include virtually any commercial material (e.g., synthetic or natural materials, such as cotton) capable of absorbing many times its weight in water. Such materials are widely available in commerce.

### Methods for Measuring Alpha-Amylase Activity

The present disclosure provides methods for use of the above-described FAM-labeled starch substrate for measuring alpha-amylase activity in a sample.

Non-limiting examples of samples include grain samples, such as cereal or pseudocereal grain samples, such as barley, oats, rice, rye, spelt, teff, triticale, wheat, rice, sorghum, millet, maize, amaranth, buckwheat, chia, quinoa, or kaniwa samples, or samples of products thereof. In various embodiments, measuring alpha-amylase activity in the sample involves preparing the sample by grinding the sample to a fine particle size. In embodiments, the particle size is such that the ground sample passes through a 12, 14, 16, 18, 20, 25, 30, 35, or 40 mesh (US standard mesh dimensions) sieve with less than 5% retain by mass. In some instances, it is advantageous for the particle size to be such that the ground sample passes through a 20 mesh (US standard mesh dimensions) sieve with less than 5% retain.

In an exemplary embodiment, a method for measuring alpha-amylase activity in the sample involves preparing a mixture containing water, the sample (e.g., the sample ground as described above), and the FAM-labeled starch substrate. The FAM-labeled starch substrate can be added to the aqueous solution in the form of tablets prepared as described above. The water can be distilled water, distilled-deionized water, MilliQ water, tap water, well water, or any water available at a site at which the method is to be carried out. The method involves mixing the mixture to yield a slurry containing the sample and the FAM-labeled starch substrate. In various embodiments, the mixture contains about or at least about 0.01 g/mL, 0.05 g/mL, 0.1 g/mL, 0.2 g/mL, 0.3 g/mL, 0.4 g/mL, 0.5 g/mL, or 1 g/mL of the sample. In some cases, the mixture contains no more than about 0.05 g/mL, 0.1 g/mL, 0.2 g/mL, 0.3 g/mL, 0.4 g/mL, 0.5 g/mL, or 1 g/mL of the sample. In embodiments, the mixture contains about or at least about 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 10 mg/mL, 20 mg/mL, 30 mg/mL, 40 mg/mL, 50 mg/mL, 100 mg/mL, 200 mg/mL, 300 mg/mL, 400 mg/mL, 500 mg/mL, or 1 g/mL. of the FAM-labeled starch substrate. In embodiments, the mixture contains less than 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 10 mg/mL, 20 mg/mL, 30 mg/mL, 40 mg/mL, 50 mg/mL, 100 mg/mL, 200 mg/mL, 300 mg/mL, 400 mg/mL, 500 mg/mL, or 1 g/mL of the FAM-labeled starch substrate.

The method further involves incubating the mixture for a period of about or at least about 10 seconds, 20 seconds, 30 seconds, 1 minute, 2 minutes, 3 minutes, 4 minutes, or 5 minutes. The method may involve incubating the mixture for a period of no more than about 10 seconds, 20 seconds, 30 seconds, 1 minute, 2 minutes, 3 minutes, 4 minutes, 5 minutes, 6 minutes, 7 minutes, 8 minutes, 9 minutes, or 10 minutes. The incubation may be carried out at room temperature and/or at a temperature of about or at least about 20° C., 25° C., 30° C., 35° C., 40° C., 50° C., 55° C., 60° C., 65° C., 70° C., 75° C., or 80° C. (e.g., 60° C.). The incubation may be carried out at room temperature and/or at a temperature of no more than about 20° C., 25° C., 30° C., 35° C., 40° C., 50° C., 55° C., 60° C., 65° C., 70° C., 75° C., or 80° C. In some cases, it is advantageous to pre-heat the water to the incubation temperature prior to adding the sample and/or FAM-labeled starch substrate to the water.

Following the incubation period, the method may involve separating the FAM-labeled starch substrate and the sample from the soluble fraction of the mixture. The separation may be carried out by any suitable method available in the art, such as through filtration or centrifugation. The method in various embodiments involves measuring fluorescence in the resulting solution containing only the soluble fraction of the mixture and then using the fluorescence measurement to calculate a predicted Falling Numbers value for the sample or other quantitative indicator of alpha amylase activity in the sample. Fluorescence can be measured using any suitable method available in the art, such as the use of a fluorimeter set for an excitation wavelength of about 485 nm and an emission wavelength of about 516 nm.

In another embodiment, following the incubation period, levels of soluble starch fragments covalently linked to a detectable label (e.g., fluorescein) are measured in the mixture with or without separation of the soluble fraction of the mixture from the insoluble fraction of the mixture using a lateral flow device (LFD) of the disclosure. The LFD is placed into the mixture or soluble fraction separated therefrom and allowed to run for about or at least about 30 sec, 1 min, 2 min, 3 min, 4 min, 5 min, 6 min, 7 min, 8 min, 9 min, or 10 min. In some cases, the LFD is allowed to run for no more than about 1 min, 2 min, 3 min, 4 min, 5 min, 6 min, 7 min, 8 min, 9 min, or 10 min. Then, the LFD is read using a device suitable for reading the LFD (e.g., a QuickScan reader) and the data obtained through the read is used to calculate a predicted Falling Numbers value for the sample or other quantitative indicator of alpha amylase activity in the sample.

In some embodiments, the method involves an extraction step. In such methods, the sample is mixed with the water to yield a mixture followed by separation of the aqueous portion of the mixture from the insoluble portion to yield an extract before adding the FAM-labeled starch substrate to the extract, or a portion thereof. In some cases, the sample is mixed in the water for a period of about or at least about 15 sec, 30 sec, 45 sec, 1 min, 2 min, 3 min, 4 min, or 5 min prior to the removal of the sample from the water to yield an extract. In some cases, the sample is mixed in the water for a period of no more than about 15 sec, 30 sec, 45 sec, 1 min, 2 min, 3 min, 4 min, or 5 min prior to the removal of the sample from the water to yield an extract.

In embodiments, the water is at a temperature of about or at least about 20° C., 25° C., 30° C., 35° C., 40° C., 50° C., 55° C., 60° C., 65° C., 70° C., 75° C., or 80° C. (e.g., about 60° C. or 70° C.) during the mixing. In some cases, the water is at a temperature of no more than about 20° C., 25° C., 30° C., 35° C., 40° C., 50° C., 55° C., 60° C., 65° C., 70° C., 75° C., or 80° C. during the mixing. In various embodiments, the water contains about or at least about 0.01 g/mL, 0.05 g/mL, 0.1 g/mL, 0.2 g/mL, 0.3 g/mL, 0.4 g/mL, 0.5 g/mL, or 1 g/mL of the sample. In some embodiments, the mass to volume ratio of sample to water in the sample-water mixture is about or at least about 1:2, 1:3, 1:4, 1:5, 1:6, 1:10, or 1:20. In some cases, the water contains no more than about 0.05 g/mL, 0.1 g/mL, 0.2 g/mL, 0.3 g/mL, 0.4 g/mL, 0.5 g/mL, or 1 g/mL of the sample. In embodiments, the FAM-labeled starch substrate is added to the extract in an amount of about or at least about 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 10 mg/mL, 20 mg/mL, 30 mg/mL, 40 mg/mL, 50 mg/mL, 100 mg/mL, 200 mg/mL, 300 mg/mL, 400 mg/mL, 500 mg/mL, or 1 g/mL. of the FAM-labeled starch substrate. In embodiments, the FAM-labeled starch substrate is added to the extract in an amount of no more than about 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 10 mg/mL, 20 mg/mL, 30 mg/mL, 40 mg/mL, 50 mg/mL, 100 mg/mL, 200 mg/mL, 300 mg/mL, 400 mg/mL, 500 mg/mL, or 1 g/mL of the FAM-labeled starch substrate.

In embodiments, the method involves incubating the mixture containing the extract and the FAM-labeled starch substrate for a period of about or at least about 10 sec, 15 sec, 20 sec, 25 sec, 30 sec, 35 sec, 40 sec, 45 sec, 50 sec, 55 sec, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, or 10 min. In some cases, the time is less than 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, or 10 min. In some cases, the method involves incubating the mixture containing the extract and the FAM-labeled starch substrate for a period of about 30 seconds. In embodiments, the incubation is carried out at a temperature of about or at least about 20° C., 25° C., 30° C., 35° C., 40° C., 50° C., 55° C., 60° C., 65° C., 70° C., 75° C., or 80° C. (e.g., 60° C.). In some cases, the temperature is less than about 20° C., 25° C., 30° C., 35° C., 40° C., 50° C., 55° C., 60° C., 65° C., 70° C., 75° C., or 80° C. In some instances, the temperature is about 70° C. In some embodiments, the method further involves stopping the reaction of the FAM-labeled starch substrate with alpha amylase in the extract (e.g., by adding DB6 buffer available from EnviroLogix (catalog no. KR-268-11, part no. 12452, by raising the pH of the solution (e.g., to about 8, 9, or 10), and/or by adding sodium tetraborate to the solution) to the extract). The active ingredient in DB6 is sodium tetraborate, and the pH of the DB6 buffer is about 9. In various embodiments, sufficient sodium tetraborate is added to the solution to reach a final molarity of about or at least about 0.005 M, 0.01 M, 0.05 M, 0.1 M, 0.2 M, 0.3 M, 0.4 M, or 0.5 M. In some embodiments, sufficient sodium tetraborate is added to the solution to reach a final molarity of no more than about 0.01 M, 0.05 M, 0.1 M, 0.2 M, 0.3 M, 0.4 M, or 0.5 M.

In some embodiments, the method involves separating the extract from the sample. In some embodiments, the method does not involve separating the extract from the sample. Separation of the extract from the sample may be carried out using any methods available to the skilled practitioner, such as settling, centrifugation, or filtration. In some embodiments, the method does not involve filtration or centrifugation. In embodiments, the method involves separating the extract from the sample by allowing the mixture containing the sample to settle for about or at least about 10 seconds, 15 seconds, 20 seconds, 25 seconds, 30 seconds, 35 seconds, 40 seconds, 45 seconds, 50 seconds, 55 seconds, 1 min, 2 min, 3 min, 4 min, 5 min, 6 min, 7 min, 8 min, 9 min, or 10 min. In some instances, for the mixture containing the sample is allowed to settle for about 30 seconds.

In embodiments, following the incubation, fluorescence is measured in the resulting incubated solution. The fluorescence measurement may be used to calculate a predicted Falling Numbers value for the sample or other quantitative indicator of alpha amylase activity in the sample. Fluorescence can be measured using any suitable method available in the art, such as the use of a fluorimeter set for an excitation wavelength of about 485 nm and an emission wavelength of about 516 nm.

In another embodiment, following the incubation, the LFD of the disclosure is placed into the incubated solution and allowed to run for about or at least about 30 sec, 1 min, 2 min, 3 min, 4 min, 5 min, 6 min, 7 min, 8 min, 9 min, or 10 min. In some cases, the LFD is allowed to run for no more than about 1 min, 2 min, 3 min, 4 min, 5 min, 6 min, 7 min, 8 min, 9 min, or 10 min. Then, the LFD is read using a device suitable for reading the LFD (e.g., a QuickScan reader) and the data obtained through the read is used to calculate a predicted Falling Numbers value for the sample or other quantitative indicator of alpha amylase activity in the sample. In embodiments, reading the LFD involves measuring levels of colloidal gold present at the test line and the control line. In some cases, measuring levels of colloidal gold involves measuring color/reflectance levels at the test line and/or control line. In some embodiments, the total assay time, e.g., from the initial wheat extraction (e.g., contacting a sample with water) to the final alpha-amylase quantification using an LFD (e.g., assigning the sample a Falling Numbers value or the like and/or taking a reading from or otherwise quantifying a measurement from the lateral flow device), is about or at least about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, or 20 min. In some instances, the total assay time is less than about 9 min. The above methods provide a reliable and simple alternative to using a Falling Numbers assay. Further, in embodiments, measurements obtained through the above-describe methods correlate linearly or nearly linearly with a range of Falling Numbers values assigned to samples. In some embodiments, the correlation is over a range of Falling Numbers values of from about 100 sec, 150 sec, 200 sec, 250 sec, 300 sec, or 350 sec to about 150 sec, 200 sec, 250 sec, 300 sec, 350 sec, or 400 sec.

In various embodiments, the methods involve stopping further digestion of the FAM-labeled starch substrate through the addition of a stopping agent to the solution. In embodiments, the stopping agent is a buffer solution, such as a DB6 buffer available from EnviroLogix.

In some embodiments, the methods of the disclosure allow for measurement of greater quantitative differences in alpha-amylase activity in samples with Falling Numbers values of about 260 sec and 400 sec than commercially available substrates and/or kits (e.g., a Megazyme kit for measuring alpha-amylase activity or a Phadebas™ substrate).

### Computer Systems

The present disclosure also relates to a computer system involved in carrying out the methods of the disclosure (e.g., methods to calculate a Falling Number value for a sample based upon measurements obtained according to the methods provided herein). In embodiments, the computer is capable of outputting a predicted Falling Number value for a sample using data collected using a QuickSkan reader available from EnviroLogix analyzed using Skannex software.

A computer system (or digital device) may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code (e.g., Skannex software code) may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations. A computer can transform data into various formats for display. A graphical presentation of the results of a calculation can be displayed on a monitor, display, or other visualizable medium (e.g., a printout). In some embodiments, data or the results of a calculation may be presented in an auditory form.

In aspects, software used to analyze the data can include code that applies an algorithm to the analysis of the results. The software also can also use input data (e.g., sequence data or biochip data) to characterize cHL or PMBL.

### Kits

The invention provides kits that include a LFD for the detection of an analyte in a sample. In one embodiment, the kit includes a lateral flow device described herein. In some embodiments, the kit comprises a container, which contains the lateral flow device; such containers can be boxes, ampoules, bottles, vials, tubes, bags, pouches, blister packs, or other suitable container forms known in the art. In one embodiment, such containers may be sterile. Such containers can be made of plastic, glass, laminated paper, metal foil, or other materials suitable for holding medicaments.

If desired the device is provided together with instructions for using it to identify the presence or absence of an analyte in a sample. The instructions will generally include information about the use of the device for the identification of a particular analyte, such as an antigen in a liquid sample (e.g., environmental sample, biological sample, or liquid sample extracted from an agricultural commodity). The instructions may be printed directly on the container (when present), or as a label applied to the container, or as a separate sheet, pamphlet, card, or folder supplied in or with the container. If desired, the kit may also include a standard measure pipet, a test vial, and/or a liquid (e.g., ethanol, methanol, organic solvent, suitable buffer, such as phosphate buffered saline, or water) to be used in the extraction of a sample.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for specific embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Fluorescein (FAM)-Labeled Starch Substrate Synthesis and Post-Processing

Experiments were undertaken to prepare an insoluble fluorescein (FAM)-labeled starch substrate suitable for use in methods for detecting alpha-amylase activity in a sample (see FIG. 15).

To prepare the FAM-labeled starch substrate, a carbonyldiimidazole-activated starch substrate was first prepared. Not intending to be bound by theory, reaction of the starch with carbonyldiimidazole (CDI) activated accessible hydroxyl groups on the glucose monomers of the starch so that they were rendered amine/hydrazine reactive. 100 g commercially available corn starch was massed into a 4 L Erlenmeyer reaction flask and a stir bar was added to the flask. Then, 2000 ml anhydrous dimethyl sulfoxide (DMSO) was added to the bottle and stirred using the stir bar at 350 rpm. Separately, 100 g carbonyldiimidazole (CDI) was suspended in 300 mL DMSO and stirred with a glass rod to make a suspension. The CDI suspension was then added to the 4 L Erlenmeyer reaction flask. The reaction flask was then covered and the contents were overnight at room temperature.

In a separate flask, adipic acid dihydrazine (AAD) was conjugated fluorescein. 5 g of fluorescein disodium salt was massed into a 500 mL glass amber bottle with a stir bar. 2.622 g of AAD were then massed into the same bottle. 200 mL phosphate buffered saline (PBS) pH 6.2 was added to the vial and the resulting solution was stirred for 10 minutes using a stir bar. Then, a 50 mg/mL solution of 1-Ethyl-3-(3-dimethylaminopropyl) carbodiimide (EDC) was prepared using the PBS. 46.7 mL of the EDC solution was added to the glass amber bottle. The bottle was capped and stirred overnight at room temperature.

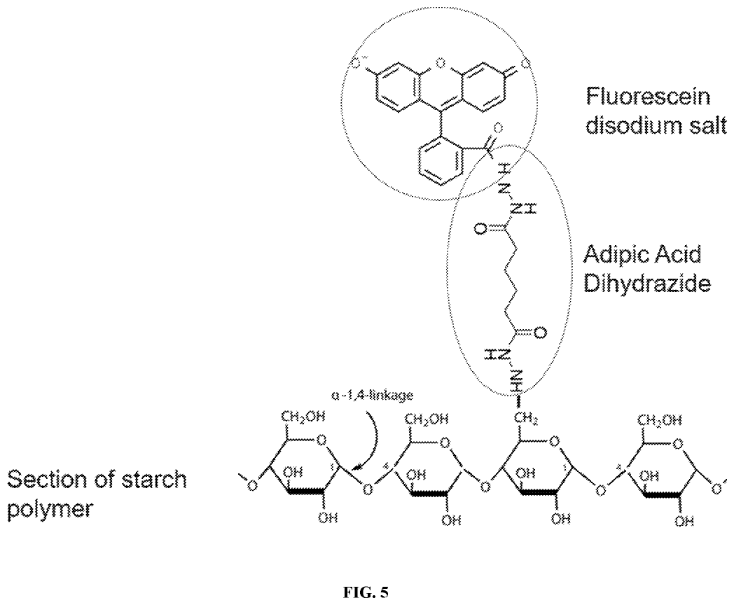

Following the overnight stirring, 20 mL milliQ® water was added to the Erlenmeyer reaction flask and 100 mL 1M sodium carbonate pH 9.6 was added to the glass amber bottle. The Erlenmeyer reaction flask was placed on a hot/stir plate and the solution was heated to 50° C. and the entirety of the contents of the glass amber bottle was then added to the Erlenmeyer reaction flask. The contents of the flask were then stirred for 2.5 hours at 50° C. Not intending to be bound by theory, during this 2.5 hour incubation period, a 1-ethyl-3-(-3-dimethylaminopropyl) carbodiimide hydrochloride (EDC) reaction took place between AAD and fluorescein to covalently link fluorescein via the carboxyl group thereof to the hydrazide of AAD and yield a product that then reacted with the activated accessible hydroxyl groups of the starch to covalently link the starch to the fluorescein (FIG. 5). The following wash steps facilitated removal of unconjugated reaction components from the FAM-labeled starch substrate. The flask was then allowed to cool to room temperature. MilliQ® water was added to the flask to bring the total reaction volume to 4 L and the resulting solution was stirred for another 45 minutes. The resulting suspension was then evenly distributed among six (6) 1 L centrifugation bottles and the total volume of solution in each bottle was brought up to 1 L using MilliQ® water. The 1 L centrifugation bottles with their contents were then centrifuged at 4000×g for 10 minutes. The resulting supernatant was then removed and the bottles were each individually refiled with 1 L milliQ® water and their contents resuspended by shaking followed by probe sonication for 5 minutes. The resulting resuspensions were each then centrifuged again at 4000×g for 10 minutes. These resuspension and centrifugation steps were repeated a total of 7 to 8 times (i.e., a total of 7 to 8 centrifugations) until no color was perceived in the supernatant. Once no color was visible in the supernatant, supernatants were removed one last time from the centrifugation bottles and their contents were resuspended in 1 L MilliQ® water, heated to 50° C. in a water bath followed and held at 50° C. for 15 minutes, and then cooled to room temperature. The centrifugation bottles and their contents were then centrifuged at 4000×g for 10 minutes. The resulting supernatants were removed and the contents of each bottle was resuspended in 500 mL MilliQ® water and the contents of pairs of centrifuge bottles were then combined in pairs to yield three centrifuge bottles each containing 1000 mL total volume of the suspensions. The three centrifuge bottles and their contents were then centrifuged at 4000×g for 10 minutes and the resulting supernatants were discarded. The contents of each of the three centrifuge vials were then resuspended in 1 L pH 7.5 Tris-Buffered Saline (TBS) containing 20 mM CaCl₂) via shaking and sonication. The TBS had the added benefit of containing primary amines that would function as a blocking feature for any remaining activated hydroxyls in the FAM-labeled starch substrate. Further, without intending to be bound by theory, calcium is a known cofactor for alpha-amylase activity, so the Ca²⁺ ions in the TBS solution may have seeded free Ca²⁺ ions within starch granules of the FAM-labeled starch substrate to later serve as a cofactor for alpha-amylase in assays using the FAM-labeled starch substrate to detect alpha-amylase activity in a sample. The three centrifuge vials and their contents were then heated in a 50° C. water bath for 15 minutes followed by cooling to room temperature. The three centrifuge vials and their contents were then centrifuged at 4000×g for 10 minutes, the supernatants were discarded, and the remaining contents of the three vials were resuspended in 1 L MilliQ® water using shaking and sonication. The three centrifuge vials and their contents were centrifuged again at 4000×g for 10 minutes, the supernatants discarded, and the remaining contents of the vials resuspended in 1 L absolute methanol using shaking and sonication. The resulting suspensions in methanol were suspension heated for 15 minutes at 50° C. following by cooling to room temperature and a 10-minute centrifugation at 4000×g. The contents of the three centrifuge vials were washed in methanol one last time by resuspending the contents of the vials in absolute methanol via shaking and sonication followed by heating for 15 minutes at 50° C., cooling to room temperature, and a to-minute centrifugation at 4000×g. The resulting supernatant was discarded and the resulting pellets were each resuspended in 1 L absolute methanol. The resulting suspensions were strained through four layers of cheesecloth to separate aggregated substrate from finely suspended substrate and the resulting strained solutions were then centrifuged at 4000×g for 10 minutes. The resulting supernatants were discarded and the remaining pellets were air-dried in a fume hood overnight.

Following overnight drying, the centrifuge vials were lightly shaken to disturb the now-dried pellets into powder. The powder was passed through an 80-mesh sieve to yield a fine yellow powder and stored desiccated at room temperature. The resulting sieved substrate was then blended with 9× by mass of microcrystalline cellulose type 102 (MCC102) and the resulting mixture was homogenized in an Oster blender for 30 seconds on the high setting. The homogenized mixture was used to prepare tablets using an LFA tablet press, a 6 mm die, and an average tablet mass of about 80 mg. The pressure of the press was adjusted until the thickness of each tablet was 3.2 mm. The resulting tablets of FAM-labeled starch substrate were stored in 1 oz amber vials desiccated at room temperature.

The FAM-labeled starch substrate contained fluorescein covalently conjugated to a starch backbone (FIG. 5). Adipic acid dihydrazide (AAD) served as a tether between the starch and fluorescein to facilitate covalent coupling. The FAM-labeled starch substrate was free of detectable excess reagents, had a granual size comparable to that of the starch used to prepare the substrate, and was insoluble in water. Without intending to be bound by theory, washing the FAM-labeled starch substrate in both aqueous and organic solutions removed from the substrate any soluble portions of the substrate that may negatively impact the performance of the substrate in an assay for measuring alpha-amylase activity. Further, the heat steps may assist in promotion of hydration/dehydration of the FAM-labeled starch substrate granules to assist with processes including washing, blocking of activated hydroxyls (e.g., by way of the primary amine of a Tris buffer), and polysaccharide refolding. Not intending to be bound by theory, the small particle size of the FAM-labeled starch substrate led to the substrate having a large surface area accessible to alpha amylase while in suspension.

### Example 2: Use of the Fluorescein (FAM)-Labeled Starch Substrate and Commercially Available Substrates to Detect Alpha-Amylase Activity

Experiments were undertaken to demonstrate that the FAM-labeled starch substrate prepared as described in Example 1 was suitable for use in detection of alpha-amylase activity in a sample and to compare the performance of the FAM-labeled starch substrate in assays for alpha-amylase activity to that of commercially available substrates commonly used in assays for detection of alpha-amylase activity.

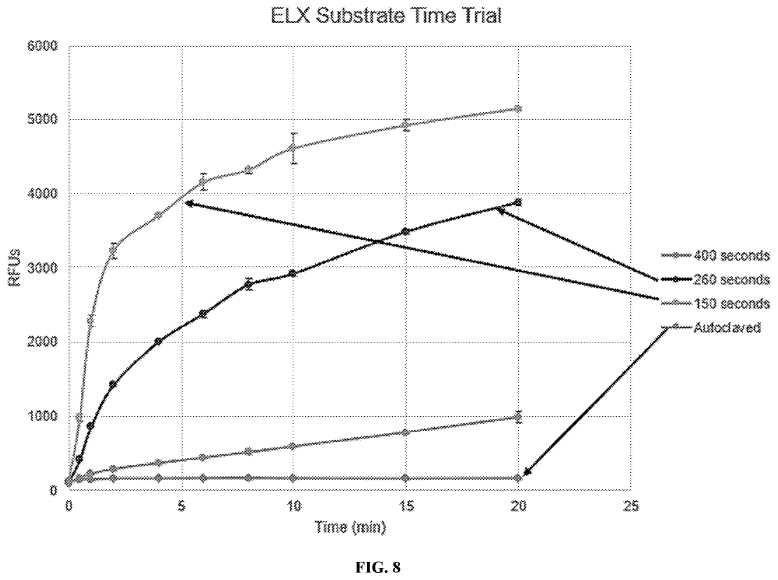

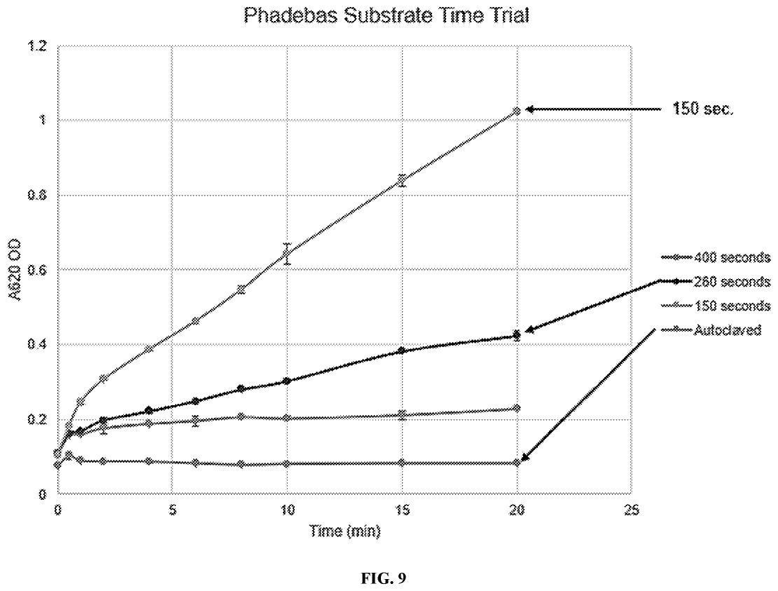

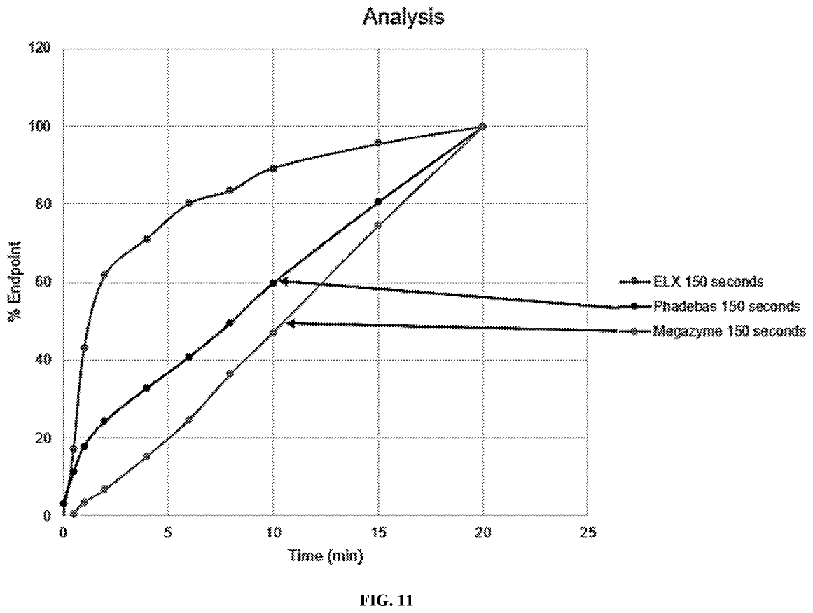

Experiments were undertaken to compare performance of the FAM-labeled starch substrate in an assay for detecting alpha-amylase activity in a sample to that of the commercially available substrate Phadebas™. Phadebas™ is a synthetic biochemical substrate containing microspheres in which a blue dye has been chemically bound and that is released when the substrate is degraded by alpha-amylase. Alpha-amylase activity was evaluated for winter wheat field samples with previously measured Falling Number assignments of about 150 sec (low Falling Number), 260 sec (medium Falling Number), and 400 sec (high Falling Number). The winter wheat field samples were all collected from field harvests and finely ground to pass through a 20 mesh sieve with less than 5% retain. A wheat sample autoclaved at 121° C. for 20 minutes was used as a negative control. Three FAM-labeled starch substrate tablets, each containing about 3 mg FAM-labeled starch substrate, or 5 Phadebas™ tablets were used for evaluating alpha-amylase activity in the winter wheat field samples. For each assay to detect alpha-amylase activity, 6 g (for Phadebas™ tablets) or 6.67 g (for FAM-labeled starch substrate) of a winter wheat field sample was massed into a 50 mL conical tube. Then, 30 mL (for Phadebas™ tables) or 33.3 mL (for FAM-labeled starch substrate) of 60° C. tap water was added to the conical tube. Substrate tablets were then added to the conical tube followed by shaking for 30 seconds to yield a wheat slurry. At set time points (e.g., 1 min, 2 min, 4 min, 6 min, 8 min, 10 min, 15 min, 20 min, where each time point was measured from the addition of the tablets to the tube), the tube was shaken and a 1 mL sample of wheat slurry was removed following shaking from the tube in duplicate and each 1 mL sample was placed in 1.5 mL centrifuge tubes. Duplicate 1 mL wheat slurry samples were also collected from the conical tube just prior to addition of the tablets (i.e., time zero (0)) and evaluated. The samples were centrifuged at 8000×g for 30 seconds. Then, 100 μL of each resulting supernatant was placed in a Microtiter plate for reading and data collection. For the FAM-labeled starch substrate, FAM fluorescence was measured using an excitation wavelength of 485 nm and an emission wavelength of 516 nm, where increasing levels of fluorescence were indicative of increasing levels of substrate digestion by alpha-amylase in the conical tube (FIG. 8). For the Phadebas™ tablets, optical absorbance was measured at a wavelength of 620 nm, where increasing levels of optical absorbance were indicative of increasing levels of substrate digestion by alpha-amylase in the conical tube (FIG. 9). The FAM-labeled starch substrate was associated with an initial rapid increase in fluorescence followed by what appeared to be a plateauing of fluorescence values at later time points (FIG. 8). The background measurement of fluorescence or optical density taken for the zero time point for the autoclaved sample was subtracted from each data point. To facilitate data comparison, curves normalized to end-point measurements were prepared for the 150 sec Falling Number samples (FIG. 11). The Phadebas™ tablets were associated with an initial brief rapid increase in optical absorbance followed by a linear increase in optical absorbance over time and no identifiable plateau at later time points. The Phadebas™ tablets allowed for less clear distinction between medium and high Falling Number samples as compared to the FAM-labeled starch substrate (compare FIGS. 8 and 9).

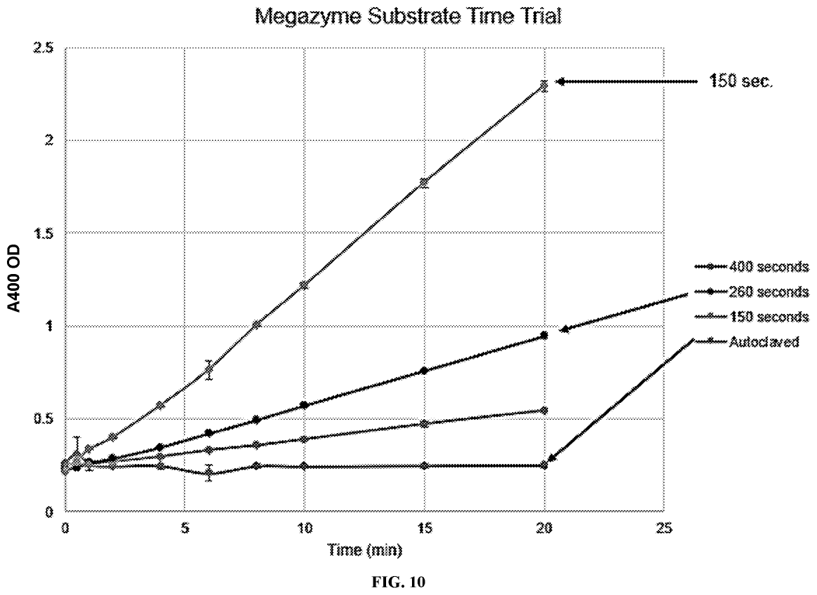

Experiments were undertaken to compare performance of the FAM-labeled starch substrate in an assay for detecting alpha-amylase activity in a sample to that of a substrate commercially available from Megazyme. The Megazyme assay was conducted using an alpha-amylase SD assay kit (Product Code: K-AMYLSD) available from Megazyme, which contained ethylidene-end blocked p-nitrophenyl maltoheptaoside as the substrate used for detecting alpha-amylase activity. Alpha-amylase activity was evaluated for winter wheat field samples with previously measured Falling Number assignments of about 150 sec, 260 sec, and 400 sec. The winter wheat field samples were all collected from field harvests and finely ground to pass through a 20 mesh sieve with less than 5% retain. A wheat sample autoclaved at 121° C. for 20 minutes was used as a negative control. Alpha-amylase activity was evaluated using the FAM-labeled starch substrate as described above. The Megazyme substrate was provided as part of a kit from Megazyme and alpha-amylase activity was evaluated using the substrate according to the instructions provided with the kit. In particular, samples were prepared by combining 0.5 g of a winter wheat field sample with 8 mL kit-provided extraction buffer in a 15 ml conical tube, which was then shaken for 30 seconds and incubated for 10 minutes in a 40° C. water bath. The conical tube was then centrifuged at 4500 g for 3 minutes and the supernatant was passed through a 1.1 μm glass pre-filter. A 0.5 mL sample was collected from the filtered supernatant to be measured as a zero timepoint. Then, the substrate from the Megazyme kit and the remaining supernatant were separately heated to 40° C. for five minutes. After being held at 40° C. for five minutes, 0.6 mL of the Megazyme kit substrate was combined with 2.4 mL of the supernatant followed by gentle shaking. The resulting mixture was incubated at 40° C. and 100 μL samples were taken in duplicate from the mixture at 30 sec, 1 min, 2 min, 4 min, 6 min, 8 min, 10 min, 15 min, and 20 min. Each collected sample was placed upon collection into 200 μL of the stop solution provided with the Megazyme kit followed by gentle mixing. Optical density readings were then measured at a 400 nm wavelength. The background measurement of fluorescence or optical density taken for the zero time point for the autoclaved sample was subtracted from each data point. To facilitate data comparison, curves normalized to end-point measurements were prepared for the 150 sec Falling Number samples (FIG. 11). The Megazyme kit substrate was associated with a linear increase in optical density over time and no identifiable plateauing of increases in optical density at later time points. The Megazyme kit substrate tablets allowed for less clear distinction between medium and high Falling Number samples as compared to the FAM-labeled starch substrate (compare FIGS. 8 and 10).

Compared to the Megazyme kit and substrate and the Phadebas™ substrates, the FAM-labeled starch substrate was associated with more rapid rates of substrate turnover (i.e., increases in measured optical density or fluorescence over time) and allowed for greater distinction between medium and high Falling Number samples.

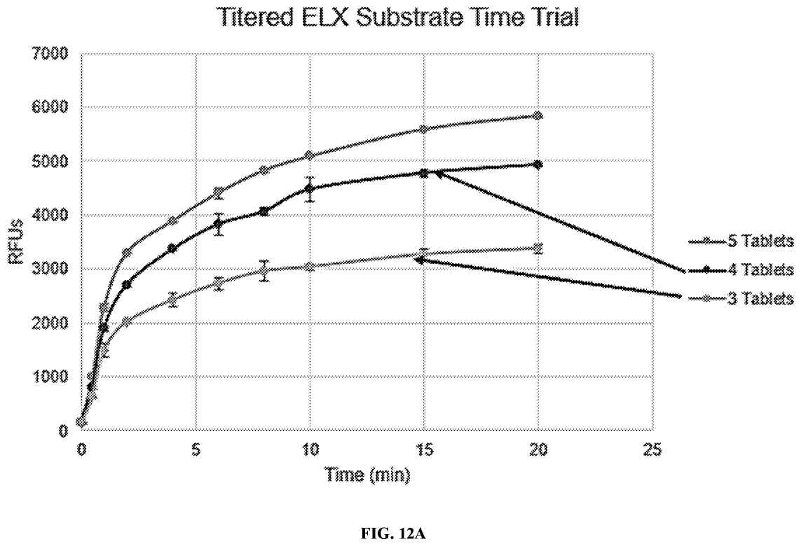

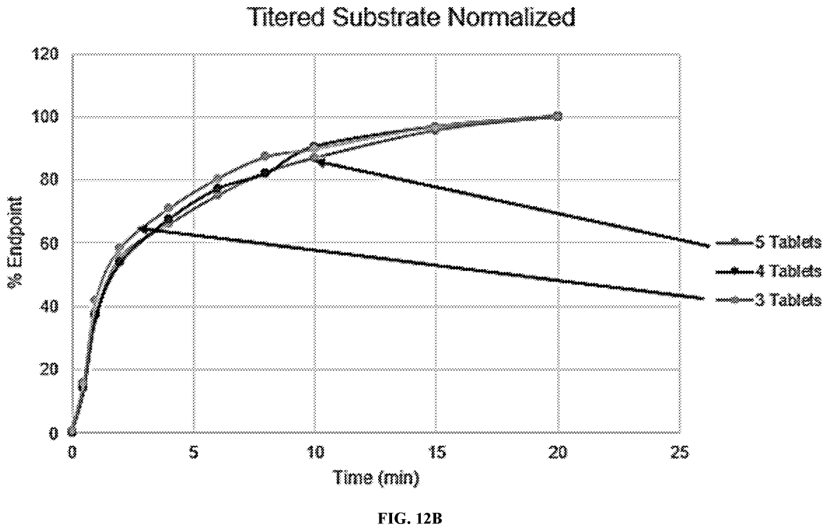

An experiment was undertaken to determine the impact of the number FAM-labeled starch substrate tablets used for detection of alpha-amylase activity in a sample on levels of detected alpha-amylase activity. Either three, four, or five tablets were used to detect alpha-amylase activity in the 150 sec falling number winter wheat field sample according to the method described above (FIGS. 12A and 12B). The performance of the FAM-labeled starch substrate was consistent across the different amounts of substrate used. The amount of the substrate used to detect alpha-amylase activity did not impact rates of substrate digestion (i.e., rate of increase in fluorescence) (FIG. 12B). Further, substrate was associated with rapid digestion rates. Not intending to be bound by theory or mode of operation, the small size of the FAM-labeled starch substrate particles (on the order of 20 micrometers) facilitated homogeneity of the tablets allowing for tablet size flexibility and consistent performance.

### Example 2: Fluorescein (FAM)-Labeled Starch Substrate Imaging

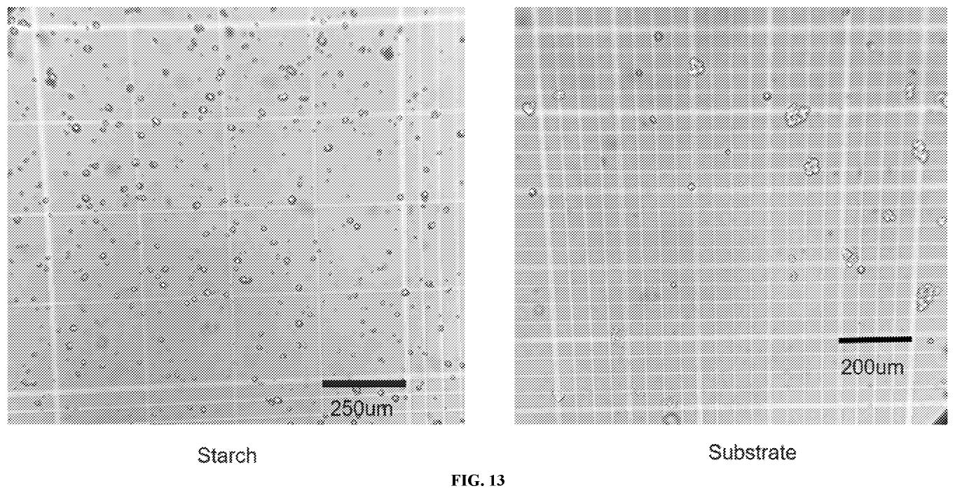

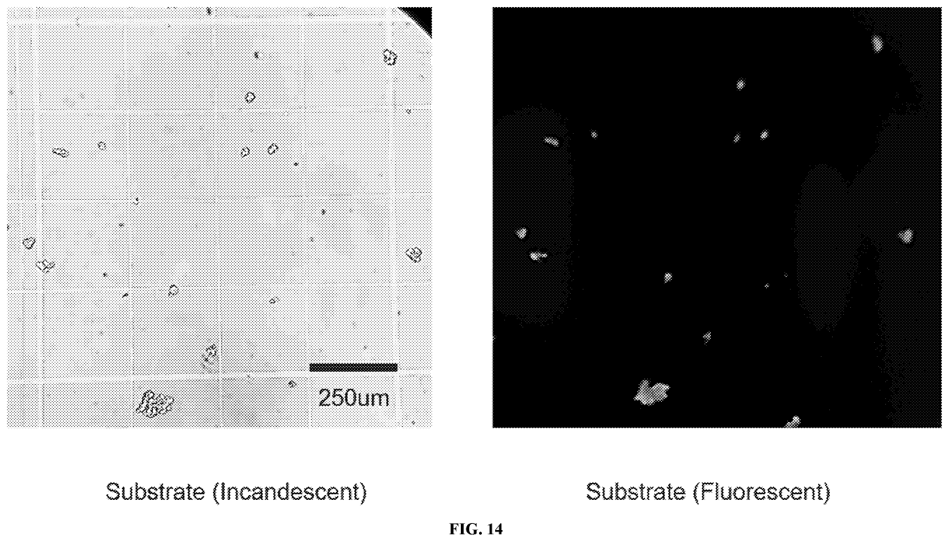

Experiments were undertaken to characterize the FAM-labeled starch substrate through microscopic imaging. Suspensions were prepared in MilliQ® of both the starch used to prepare the FAM-labeled starch substrate (0.5% mass/volume) and the FAM-labeled starch substrate (0.1% mass/volume). Ten μL of each suspension was placed onto a hemocytometer and viewed using a Nikon inverted microscope. First, images were taken at 100× magnification using standard visible light imaging with an incandescent lamp (FIG. 13). The granule size was comparable between the starch used to prepare the substrate and the FAM-labeled starch substrate. Next, images were taken by fluorescent imaging using a blue LED 470 nm lamp above hemocytometer and a 580 nm amber filter beneath the hemocytometer. The fluorescent imaging demonstrated a homogenous distribution of fluorescent dye covalently conjugated to the starch backbone of the FAM-labeled starch substrate (FIG. 14). The FAM-labeled starch granules were nearly equivalent in size to plan starch granules and, therefore, representative of what an alpha-amylase enzyme would encounter naturally in a wheat kernel and Falling Numbers test.

### Example 4: Fluorescence-Reporting Substrate Assay

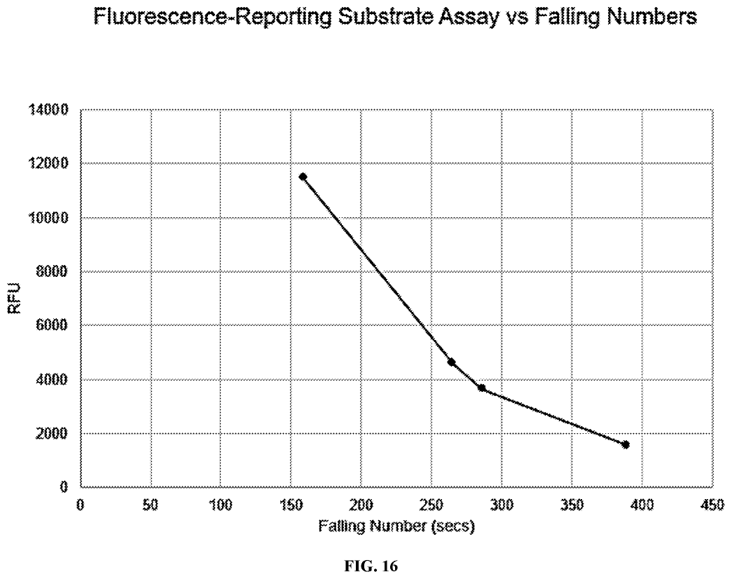

An experiment was undertaken to demonstrate the use of the FAM-labeled starch substrate in a fluorescence-reporting substrate assay for detection of alpha-amylase activity in a sample. The samples evaluated were soft white winter wheat filed samples having pre-assigned Falling Number values. To measure alpha-amylase activity in a sample, 5 g of finely ground soft winter wheat berries was placed into a 50 ml conical tube containing FAM-labeled starch substrate tablets prepared as described in Example 1. Then, 25 mL of 60° C. pre-warmed tap water was added to the conical tube, which was then capped and shaken for 30 seconds. The resulting slurry was allowed to incubate and settle for an additional 30 seconds. About 1.5 mL of the top liquid phase was transferred to a 2 mL microfuge tube and then centrifuged at 8000×g for 30 seconds. The centrifugation led to a separation of insoluble undigested FAM-labeled starch substrate from fluorescently labeled soluble products of digestion of the substrate and served to effectively halt the substrate digestion reaction within the supernatant. The resulting supernatant was then carefully removed from the tube and fluorescence of the supernatant was measured using an excitation wavelength of 485 nm and an emission wavelength of 516 nm, where increasing levels of fluorescence were indicative of increasing levels of substrate digestion by alpha-amylase (FIG. 16). The resulting fluorescence measurements demonstrated a strong correlation between fluorescence readings and Falling Numbers assignments for the samples, thereby showing that the FAM-labeled starch substrate can be effectively used in a simple and rapid assay for measuring alpha-amylase activity in samples.

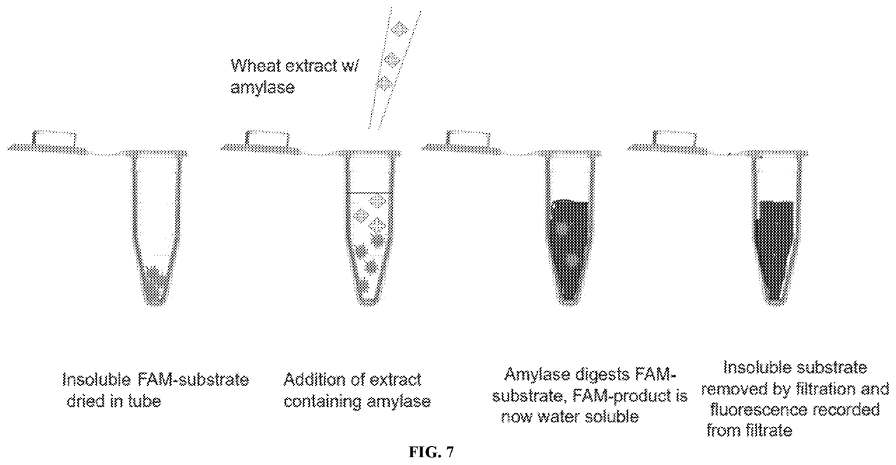

In another Experiment, alpha amylase activity was measured according to the method of FIG. 7. First, 20 g of ground wheat was mixed with 100 mL water followed by shaking for 1 minute. The resulting slurry was then transferred to a conical centrifugation tube and centrifuged at 2000×g for 30 seconds. A 3 mL sample of the resulting supernatant was placed in an incubator tube containing a FAM-labeled starch substrate pellet and incubated at 50° C. for 5 minutes. Then, the contents of the incubator tube were filtered using a 0.45 μm filter and 50 μL of the resulting filtrate was placed into an optical 0.2 mL microfuge tube and fluorescence of the filtrate was measured using an 8-tube strip fluorimeter equipped with excitation/emission optics at 493 nm/517 nm, respectively.

### Example 5: Lateral Flow Device (LFD)-Reporting Substrate Assay

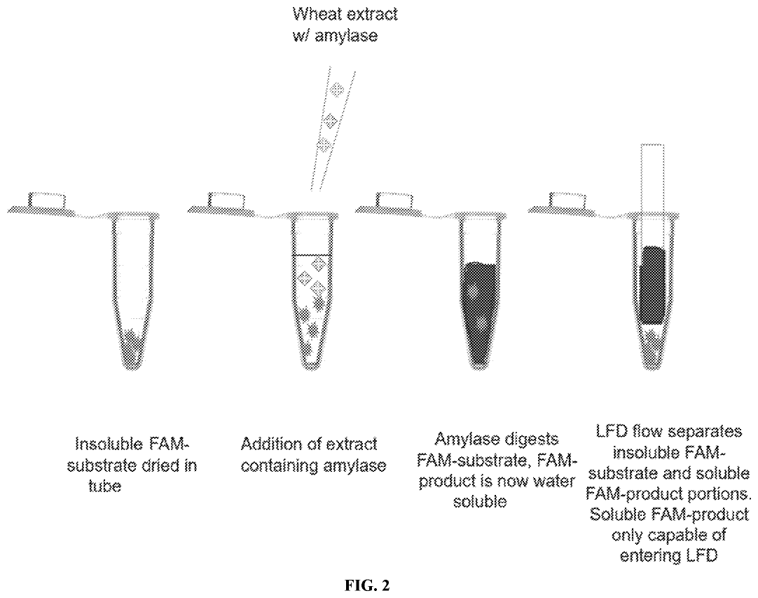

Experiments were undertaken to use a lateral flow device in a lateral flow device-reporting substrate assay involving the use of the FAM-labeled starch substrate (e.g., FIG. 2).

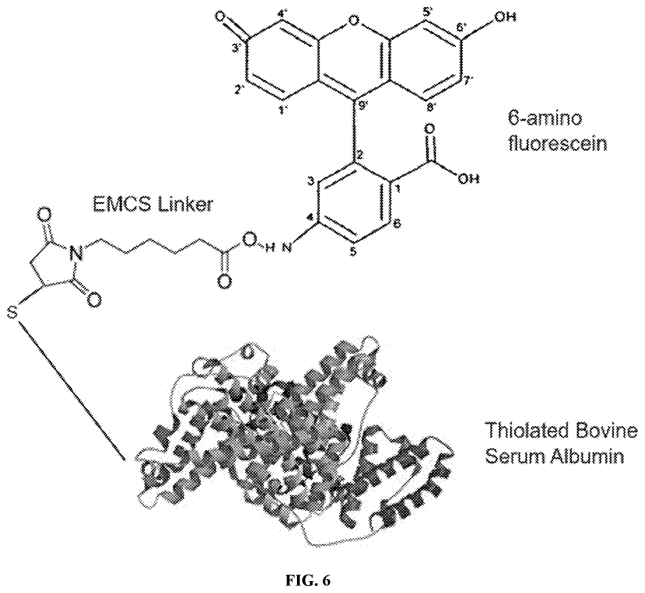

A lateral flow device (LFD) was first prepared for use in the LFD-reporting substrate assay (FIG. 1). To prepare the LFD, a 25 mm long CN140 nitrocellulose strip (Sartorius) was sprayed with a PBS solution containing fluorescein conjugated to bovine serum albumin (BSA) as a capture conjugate (FAM-BSA capture conjugate; see FIG. 6) to yield a test line (TL) of the LFD (FIGS. 1 and 3). The BSA conjugated to fluorescein was prepared by thiolating BSA and 6-amino fluorescein was allowed to react with 6-Maleimidohexanoic acid N-hydroxysuccinimide ester (EMCS) in a buffer to prepare 6-amino fluorescein covalently linked to EMCS, then the fluorescein linked to the EMCS was combined with the tiolated BSA and allowed to react to yield the FAM-BSA capture conjugate. Next, a PBS solution containing rabbit anti-mouse polyclonal antibody was sprayed onto the CN140 nitrocellulose to yield a control line (FIGS. 1 and 3). The nitrocellulose strip was then allowed to dry overnight at 45° C. The anti-fluorescein isothiocyanate (FITC) monoclonal antibody 1F11 (Jackson Immunoresearch Laboratories catalog no. 200-002-037) was conjugated to colloidal gold nanoparticles under passive conjugation conditions used with citrate-reduced gold particles to yield a gold-antibody conjugate. The 1F11 monoclonal antibody was classified as “IgG fraction” and believed to be associated with cell line 1F8-1E4. A solution containing 60% by weight of a concentrated solution of gold-antibody conjugate and 40% by weight sucrose was sprayed onto a 30 mm long fibrous polyester laminating pad strip previously treated with Triton-X100 and bovine serum albumin and allowed to dry at 45° C. for 2 hours. The lateral flow device was then assembled as shown in FIG. 1 by adhering the fibrous polyester laminating pad strip, the nitrocellulose strip, and a 39 mm long absorbent cellulose-based pad with a product-specific barcode to a 90 mm adhesive card that was then cut into 3.88 mm strips to yield anti-fluorescein LFDs for use in the LFD-reporting substrate assay for use in measuring alpha-amylase activity in a sample. The LFD devices were stored in desiccated tubes at 4° C.

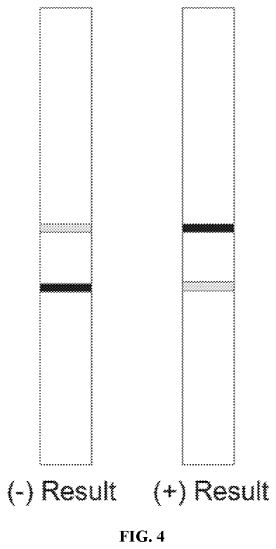

FIGS. 3 and 4 provides a schematic diagrams illustrating the operation of the LFD. The LFD mediated an immunoassay. The assay target was a starch fragment covalently linked to fluorescein. Degradation of the FAM-labeled starch substrate by alpha-amylase liberates water-soluble starch fragments covalently linked to fluorescein (e.g., fluorescein-glucose molecules) that may be detected using the LFD. Upon administration of a sample containing the assay target to the LFD, the assay target bound with the gold-antibody conjugate, which prevented later test line binding by the gold-antibody conjugate. Following administration of a sample, the LFD strips were evaluated using a QuickScan strip reader equipped with Skannex software. The QuickScan strip reader measured levels of colloidal gold particle present at the test line and control line by detecting levels of color/reflectance. A signal intensity calculation based on test and control line intensities was backfitted to a calibration curve of wheat samples with known Falling Number values and a projected Falling Number value was calculated. The calibrated curve could be encoded into a multi-matrix bar code supplied with a kit containing the LFD and made available through a TotalHub data portal available at EnviroLogix.

The LFDs were used in an assay to detect alpha amylase activity in a wheat sample. First, 20 g of ground wheat was mixed with 100 mL water followed by shaking for 1 minute. The resulting slurry was transferred to a conical centrifuge tube and centrifuged at 2000×g for 30 seconds. Then, 300 μL of the resulting supernatant was placed in an incubator tube containing a FAM-labeled starch substrate tablet prepared as described in Example 1 and allowed to incubate at 50° C. for 5 minutes. Following the 5-minute incubation, the incubation tube was removed from the heat and 300 μL of DB6 buffer available from EnviroLogix (catalog no. KR-268-11, part no. 12452) was added to the tube. The tube was then vortexed briefly and a LFD placed into the tube and run for 5 minutes. The sample pad (see FIG. 1) was severed from the LFD and fluorescence was measured at the TL and CL using a QuickScan strip reader as described above.

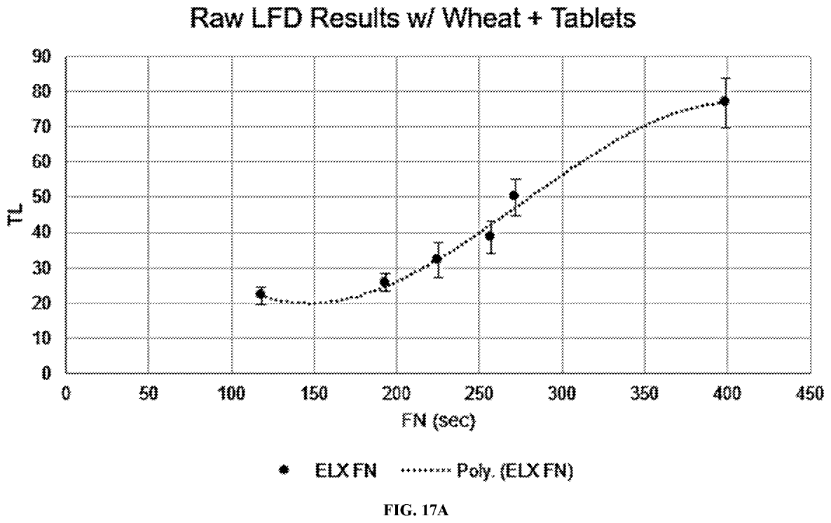

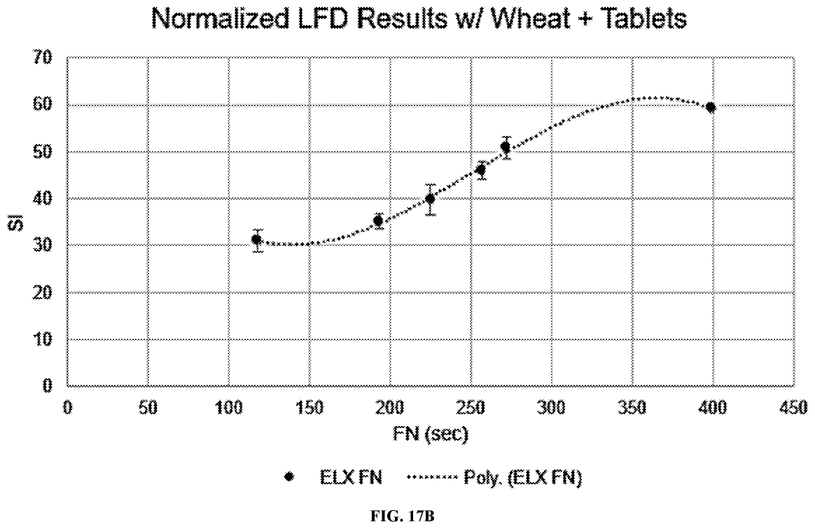

Another experiment was undertaken to demonstrate the ability of the LFDs to be used for detection of alpha-amylase activity in a sample using LFD-reporting substrate assay. Soft white winter wheat field samples of various predetermined Falling Numbers values were used as samples in the experiment. All samples were finely ground to pass through a 20 mesh sieve with less than 5% by weight retain. One gram of each wheat sample was then extracted by adding 5 mL of tap water to the sample in a tube followed by shaking for 30 seconds and centrifugation at 2000×g for 30 seconds. 300 μL of the resulting supernatant was then added to a 12 mm×75 mm polypropylene tube and 1 tablet containing about 2 mg of the FAM-labeled starch substrate was placed into the tube followed by light shaking until the FAM-labeled starch substrate became dispersed in the supernatant. The tube was then placed into a 50° C. heat block and incubated for 5 minutes. Following the 5 minute incubation, 300 μL of DB6 buffer available from EnviroLogix (catalog no. KR-268-11, part no. 12452) was added to the tube followed by light shaking and filtration of the resulting solution through a 0.22 μm filter. A LFD prepared as described above was placed into the resulting filtrate and allowed to run for 5 minutes. The lower portion of the LFD beneath the nitrocellulose was then severed to stop flow and the LFD was read on a QuickSkan reader equipped with Skannex software. The resulting data demonstrated a correlation between LFD measurements and Falling Numbers values (FIGS. 17A and 17B).

These results demonstrate that the LFD can be used in combination with the FAM-labeled starch substrate to detect alpha-amylase activity in a sample as a reliable and simple alternative to using a Falling Numbers assay.

### Example 6: Fluorescein (FAM)-Labeled Starch Substrate Synthesis and Post-Processing

An experiment was undertaken to prepare the starch substrate conjugated to insoluble fluorescein (FAM) suitable for use in methods for detecting alpha-amylase activity in a sample.

To prepare the FAM-labeled starch substrate, a carbonyldiimidazole-activated starch substrate was first prepared. Not intending to be bound by theory, reaction of the starch with carbonyldiimidazole (CDI) activated accessible hydroxyl groups on the glucose monomers of the starch so that they were rendered amine/hydrazine reactive. 100 g commercially available corn starch was massed into a 4 L Erlenmeyer reaction flask and a stir bar was added to the flask. Then, 2000 ml anhydrous dimethyl sulfoxide (DMSO) was added to the bottle and stirred using the stir bar at 350 rpm. Separately, 100 g carbonyldiimidazole (CDI) was suspended in 300 mL DMSO and stirred with a glass rod to make a suspension. The CDI suspension was then added to the 4 L Erlenmeyer reaction flask. The reaction flask was then covered and the contents were stirred overnight at room temperature.

In a separate flask, adipic acid dihydrazine (AAD) was conjugated to fluorescein. 10 g of fluorescein disodium salt was massed into a 500 mL glass amber bottle with a stir bar. 5.244 g of AAD were then massed into the same bottle. 200 mL phosphate buffered saline (PBS) pH 6.2 was added to the vial and the resulting solution was stirred for 10 minutes using a stir bar. Then, a 50 mg/mL solution of 1-Ethyl-3-(3-dimethylaminopropyl) carbodiimide (EDC) was prepared using the PBS. 93.4 mL of the EDC solution was added to the glass amber bottle. The bottle was capped and stirred overnight at room temperature.

Following the overnight stirring, 20 mL milliQ® water was added to the Erlenmeyer reaction flask and 100 mL 1M sodium carbonate pH 9.6 was added to the Erlenmeyer flask. The Erlenmeyer reaction flask was placed on a hot/stir plate and the solution was heated to 50° C. and the entirety of the contents of the glass amber bottle was then added to the Erlenmeyer reaction flask. The contents of the flask were then stirred for 2.5 hours at 50° C. Not intending to be bound by theory, during this 2.5 hour incubation period, a 1-ethyl-3-(-3-dimethylaminopropyl) carbodiimide hydrochloride (EDC) reaction took place between AAD and fluorescein to covalently link fluorescein via the carboxyl group thereof to the hydrazide of AAD and yield a product that then reacted with the activated accessible hydroxyl groups of the starch to covalently link the starch to the fluorescein (FIG. 5). The following wash steps facilitated removal of unconjugated reaction components from the FAM-labeled starch substrate. The flask was then allowed to cool to room temperature. MilliQ® water was added to the flask to bring the total reaction volume to 4 L and the resulting solution was stirred for approximately 1 hour until bubble formation is no longer observed. The resulting suspension was then evenly distributed among six (6) 1 L centrifugation bottles and the total volume of solution in each bottle was brought up to 1 L using MilliQ® water. The 1 L centrifugation bottles with their contents were then centrifuged at 4000×g for 10 minutes. The resulting supernatant was then removed, and the bottles were each individually refiled with 1 L milliQ® water and their contents resuspended by shaking followed by probe sonication for 5 minutes. The resulting resuspensions were each then centrifuged again at 4000×g for 10 minutes. These resuspension and centrifugation steps were repeated a total of 7 to 8 times (i.e., a total of 7 to 8 centrifugations) until no color was perceived in the supernatant. Last refill and resuspension with water heated to 50C in water bath for 15 min, cooled to room temperature and centrifuged again.

Once no color was visible in the supernatant, supernatants were removed one last time from the centrifugation bottles and their contents were resuspended in 1 L MilliQ® water, heated to 50° C. in a water bath followed and held at 50° C. for 15 minutes, and then cooled to room temperature. The centrifugation bottles and their contents were then centrifuged at 4000×g for 10 minutes. The resulting supernatants were removed and the contents of each bottle was resuspended in 500 mL MilliQ® water and the contents of pairs of centrifuge bottles were then combined in pairs to yield three centrifuge bottles each containing 1000 mL total volume of the suspensions. The three centrifuge bottles and their contents were then centrifuged at 4000×g for 10 minutes and the resulting supernatants were discarded. The contents of each of the three centrifuge vials were then resuspended in 1 L pH 7.5 Tris-Buffered Saline (TBS) containing 20 mM CaCl₂) via shaking and sonication. The TBS had the added benefit of containing primary amines that would function as a blocking feature for any remaining activated hydroxyls in the FAM-labeled starch substrate. Further, without intending to be bound by theory, calcium is a known cofactor for alpha-amylase activity, so the Ca²⁺ ions in the TBS solution may have seeded free Ca²⁺ ions within starch granules of the FAM-labeled starch substrate to later serve as a cofactor for alpha-amylase in assays using the FAM-labeled starch substrate to detect alpha-amylase activity in a sample. The three centrifuge vials and their contents were then heated in a 50° C. water bath for 15 minutes followed by cooling to room temperature. The three centrifuge vials and their contents were then centrifuged at 4000×g for 10 minutes, the supernatants were discarded, and the remaining contents of the three vials were resuspended in 1 L MilliQ® water using shaking and sonication. The three centrifuge vials and their contents were centrifuged again at 4000×g for 10 minutes, the supernatants discarded, and the remaining contents of the vials resuspended in 1 L absolute methanol using shaking and sonication. The resulting suspensions in methanol were suspension heated for 15 minutes at 50° C. following by cooling to room temperature and a 10-minute centrifugation at 4000×g. The contents of the three centrifuge vials were washed in methanol one last time by resuspending the contents of the vials in absolute methanol via shaking and sonication followed by heating for 15 minutes at 50° C., cooling to room temperature, and a to-minute centrifugation at 4000×g. The resulting supernatant was discarded and the resulting pellets were each resuspended in 1 L absolute methanol. The resulting suspensions were strained through four layers of cheesecloth to separate aggregated substrate from finely suspended substrate and the resulting strained solutions were then centrifuged at 4000×g for 10 minutes. The resulting supernatants were discarded and the remaining pellets were air-dried in a fume hood overnight.

Following overnight drying, the centrifuge vials were lightly shaken to disturb the now-dried pellets into powder. The powder was passed through an 80-mesh sieve to yield a fine yellow powder and stored desiccated at room temperature. The resulting sieved substrate was then blended with 9× by mass of microcrystalline cellulose type 102 (MCC102) binder and the resulting mixture was homogenized in an Oster blender for 15 seconds on the high setting. The homogenized mixture was used to prepare tablets using an LFA tablet press, a 6 mm die, and an average tablet mass of about 80 mg. The pressure of the press was adjusted until the thickness of each tablet was 3.2-3.4 mm. The resulting tablets of FAM-labeled starch substrate were stored in 1 oz amber vials and desiccated at 4° C. until use.

### Example 7: Use of the Fluorescein (FAM)-Labeled Starch Substrate and Commercially Available Substrates to Detect Alpha-Amylase Activity

Experiments were undertaken to use the FAM-labeled starch substrate prepared in Example 6 to detect alpha-amylase activity in a lateral flow device-based assay.

To assist in preparing a standard curve, samples with predetermined Falling Numbers assignments were prepared. First, stock soft white winter wheat was partially germinated to achieve a Falling Numbers value of approximately 100 seconds. Wheat berries were evenly spread across 18′×24′ trays that were lined with water-saturated paper towels. The trays were covered with plastic wrap and placed at 4° C. for 48 hours void of light. The trays were then moved to 22° C. and left for 12 hours void of light. After, the wheat was placed onto new trays with dry paper towels and air-dried in a fume hood for 24 hours to yield cold-stratified wheat. Next, the cold-stratified wheat and the stock soft white winter wheat were each separately ground twice using a Bunn grinder on Turkish setting and sieved through a 20 mesh (0.8 mm) sieve. Various blends of cold-stratified wheat and stock wheat were made to achieve a range of Falling Numbers values between 150 and 400 seconds. The blends mixed thoroughly. Falling Numbers values for each blend was then measured using standard methodology.

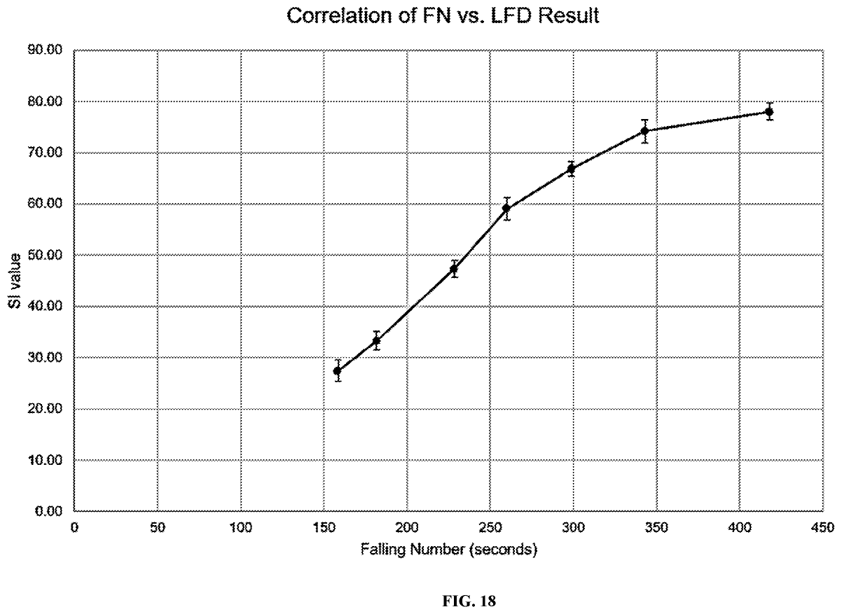

An experiment was undertaken to demonstrate the ability of the lateral flow devices (LFDs) of the disclosure to be used for the detection of alpha-amylase activity in the various blends of cold-stratified wheat and stock wheat. Five grams of each wheat sample was then extracted by adding 25 mL of MilliQ® water (5:1 ratio vol:mass of wheat sample to water) to the sample in a tube followed by shaking for 30 seconds, and centrifugation at 2000×g for 30 seconds. 300 μL of the resulting supernatant was then added to a 12 mm×75 mm polypropylene tube and 1 tablet containing about 2 mg of the FAM-labeled starch substrate was placed into the tube followed by vortexing on high for 15 seconds. The tube was then placed into a 70° C. heat block and incubated for 90 seconds. Following the 90 second incubation, 300 μL of DB6 buffer available from EnviroLogix (catalog no. KR-268-11, part no. 12452) was added directly to the tube in the incubator to stop the reaction. The tube was then removed from the incubator and placed in a rack to settle for 30 seconds. An LFD prepared as described in Example 5 was placed into the settled reaction mixture in the tube and allowed to run for 3 minutes. The lower portion of the LFD beneath the nitrocellulose was then severed to stop flow and the LFD was read on a QuickSkan reader equipped with Skannex software to provide a signal intensity value for the wheat sample. The total assay time for measuring alpha amylase activity was about 9 minutes, which is an improvement over alternative assay kits, which require approximately 10 minutes of substrate digestion and a total assay time of about 30 minutes. The resulting data demonstrated a correlation between LFD measurements and Falling Numbers values (FIG. 18).

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for producing a substrate for use in detecting alpha amylase activity in a liquid sample, said method comprising:
a) covalently linking a fluorescein molecule (FAM) to a natural starch molecule to yield a FAM-labeled starch substrate;
b) suspending the FAM-labeled starch substrate in one or more aqueous solutions one or more times, wherein each suspending is followed by separation of the FAM-labeled starch substrate from the aqueous solution; and
c) suspending the FAM-labeled starch substrate in a volatile organic solvent followed by drying of the FAM-labeled starch substrate to remove the volatile organic solvent.

2. The method of claim 1, wherein covalently linking the FAM to the natural starch molecule comprises:
a) contacting the natural starch molecule in aqueous solution with carbonyldiimidazole to activate accessible hydroxyl groups;
b) contacting the FAM with adipic acid dihydrazine (AAD) in an aqueous solution comprising 1-Ethyl-3-(3-dimethylaminopropyl) carbodiimide (EDC) so that a carboxyl group of the fluorescein becomes covalently linked to the AAD; and
c) combining the products of a) and b) to allow the AAD bound to the FAM to react with the activated accessible hydroxyl groups of the natural starch molecule to yield the FAM-labeled starch substrate.

3. The method of claim 1, wherein the natural starch is corn starch or wheat starch.

4. The method of claim 1, wherein b) comprises washing the FAM-labeled starch substrate in purified water between 5 and 10 times, wherein each washing comprises suspending the FAM-labeled starch substrate in the purified water and subsequently separating the FAM-labeled starch substrate from the purified water using centrifugation or filtration.

5. The method of claim 4, wherein at least one of the aqueous solution comprises between 2 and 50 mM Ca²⁺ ions.

6. A FAM-labeled starch substrate produced by the method of claim 1.

7. A method for measuring alpha-amylase activity in a grain sample, the method comprising:
a) preparing an aqueous mixture comprising the grain sample and the FAM-labeled starch substrate prepared according to the method of claim 1 under conditions suitable for any alpha-amylase protein present in the grain sample to degrade the FAM-labeled starch substrate and thereby produce water-soluble FAM-labeled starch fragments in the aqueous mixture;
b) separating undegraded FAM-labeled starch substrate and the grain sample from the aqueous mixture to yield a soluble fraction; and
c) quantifying the amount of FAM-labeled starch fragments in the soluble fraction.

8. The method of claim 7, wherein the concentration of the grain sample in the aqueous mixture is from about 0.1 g/mL to about 1 g/mL, and/or wherein the concentration of the FAM-labeled starch substrate added to the aqueous mixture is from about 0.2 mg/mL to about 500 mg/mL;

9. The method of claim 7, wherein b) comprises quantifying the amount of FAM-labeled starch fragments in the soluble fraction using a lateral flow device (LFD) comprising an anti-fluorescein antibody conjugated to a colloidal gold particle, a test line and a control line, wherein:
i) the test line comprises a polypeptide conjugated to fluorescein; and
ii) the control line comprises an antibody capable of binding to the anti-fluorescein antibody.

10. A method for measuring alpha-amylase activity in a grain sample, the method comprising:
a) contacting the grain sample with an aqueous solution to extract alpha-amylase polypeptides from the grain sample;
b) separating the aqueous solution from the grain sample to yield an extract solution;
c) adding the FAM-labeled starch substrate of claim 1 to the extract solution under conditions suitable for an alpha-amylase protein present in the extract solution to degrade the FAM-labeled starch substrate and thereby produce water-soluble FAM-labeled starch fragments in the extract solution; and
d) quantifying the amount of FAM-labeled starch fragments in the extract solution.

11. The method of claim 10, wherein the concentration of the grain sample in the aqueous solution is from about 0.1 g/mL to about 1 g/mL; and/or wherein the volume to mass ratio of the grain sample to the aqueous solution is from about 2:1 to 10:1.

12. The method of claim 10, wherein d) comprises quantifying the concentration of FAM in the extract solution using a fluorimeter.

13. The method of claim 12, wherein d) comprises quantifying the amount of FAM-labeled starch fragments in the extract solution using a lateral flow device (LFD) comprising an anti-fluorescein antibody conjugated to a colloidal gold particle, a test line, and a control line, wherein:
i) the test line comprises a polypeptide conjugated to fluorescein; and
ii) the control line comprises an antibody capable of binding to the anti-fluorescein antibody.

14. A kit suitable for use in the method of claim 10, wherein the kit comprises a FAM-labeled starch substrate disposed within a container.

15. A tablet comprising microcrystalline cellulose and an insoluble fluorescein-labeled starch substrate comprising a natural starch molecule covalently linked to a fluorescein molecule by an adipic acid dihydrazine linker.

16. A composition comprising a volatile organic substrate and a natural starch molecule covalently linked to a fluorescein molecule by an adipic acid dihydrazine linker.

17. A lateral flow device (LFD) comprising an anti-fluorescein antibody conjugated to a colloidal gold particle, a test line and a control line, wherein:
i) the test line comprises a polypeptide conjugated to fluorescein; and
ii) the control line comprises an antibody capable of binding to the anti-fluorescein antibody.

18. A method for producing a substrate for use in detecting alpha amylase activity in a liquid sample, said method comprising:
a) covalently linking a fluorescein molecule (FAM) to a corn starch molecule using adipic acid dihydrazide to yield a FAM-labeled starch substrate;
b) suspending the FAM-labeled starch substrate in purified water between 5 and 10 times, wherein each suspending is followed by separation of the FAM-labeled starch substrate from the purified water using centrifugation, wherein one or more of the washes of the FAM-labeled starch substrate in purified water comprises heating the purified water comprising the FAM-labeled starch substrate suspended therein to a temperature of between about 40° C. and 80° C. for between about 5 min and 30 min prior to separating the FAM-labeled starch substrate from the purified water,
c) suspending the FAM-labeled starch substrate in an aqueous solution with a pH of between 7 and 8 comprising a Tris buffer and between 2 and 50 nM Ca²⁺ ions and incubating the suspension at a temperature of between about 40° C. and 80° C. for between about 5 min and 30 min prior to separating the FAM-labeled starch substrate from the aqueous solution using centrifugation; and
d) suspending the FAM-labeled starch substrate in methanol followed by drying of the FAM-labeled starch substrate to remove the methanol.

19. A method for measuring alpha-amylase activity in a grain sample, the method comprising:
a) contacting the grain sample with an aqueous solution at a mass to volume ratio of grain sample to aqueous solution of about 5:1 for between 15 seconds and 1 minute to extract alpha-amylase polypeptides from the grain sample;
b) separating the aqueous solution from the grain sample to yield an extract solution;
c) adding between 3 mg/mL and 10 mg/mL of the FAM-labeled starch substrate of claim 71 to the extract solution and incubating the resulting mixture at a temperature of between about 65° C. and 75° C. for between 1 minute and 2 minutes under conditions suitable for an alpha-amylase protein present in the extract solution to degrade the FAM-labeled starch substrate prior to stopping the reaction to thereby produce water-soluble FAM-labeled starch fragments in the extract solution; and
d) quantifying the amount of FAM-labeled starch fragments in the extract solution using a lateral flow device (LFD) comprising an anti-fluorescein antibody conjugated to a colloidal gold particle, a test line, and a control line, wherein:
i) the test line comprises a polypeptide conjugated to fluorescein; and
ii) the control line comprises an antibody capable of binding to the anti-fluorescein antibody; and wherein the LFD further comprises:
i) a first portion comprising a site for application of a liquid sample, a liquid-permeable medium, and the anti-fluorescein antibody conjugated to the colloidal gold particle;
ii) a second portion in capillary communication with the first portion and comprising a liquid-permeable medium comprising, in order of distance from the first portion, the test line and the control line; and
iii) a third portion in capillary communication with the second portion and comprising a liquid-permeable medium.

20. The method of claim 19, wherein the method is carried out in less than 10 minutes.
